(19) United States
(12) Patent Application Publication
(10) Pub. No.: US 2017/0247693 A1
(43) Pub. Date: Aug. 31, 2017

(54) COMBINATORIAL TREATMENT OF CHEMOTHERAPY AND ARMED VIRUSES TARGETING TUMOR

(71) Applicants: Yissum Research Development Company Of The Hebrew University Of Jerusalem Ltd., Jerusalem (IL); Hadasit Medical Research Services & Development Ltd., Jerusalem (IL)

(72) Inventors: Alik HONIGMAN, Hadera (IL); Shahar FRENKEL, Tel Aviv (IL); David SHNEOR, Maale Adumim (IL)

(21) Appl. No.: 15/442,960

(22) Filed: Feb. 27, 2017

Related U.S. Application Data
(60) Provisional application No. 62/299,795, filed on Feb. 25, 2016.

(51) Int. Cl. C12N 15/113; A61K 31/655; A61K 31/7088; A61K 31/704

(52) U.S. Cl. CPC C12N 15/113; A61K 31/704; A61K 31/655; A61K 31/7088; C12N 2310/11; C12N 2310/531; C12N 2320/31; C12N 2320/32

(57) ABSTRACT

Methods, and kits for inducing cell death in proliferating cells as well as methods of treating cancer, are provided. In some embodiments, the methods comprise administering a composition comprising a replication competent retrovirus (RCR) comprising an antisense molecule that targets a hypoxia-inducible gene including but not limited to HIF-1 and CREB, and anti-cancer therapy.

### CROSS REFERENCE TO RELATED APPLICATIONS

This application claims priority to U.S. Provisional Patent Application No. 62/299,795, filed Feb. 25, 2016, the contents of which are incorporated herein by reference in their entirety.

### FIELD OF INVENTION

The preset invention is related to compositions comprising antisense molecules targeting hypoxia-related control genes combined with anti-cancer therapy for inhibiting proliferating cells and treatment of cancer.

### BACKGROUND OF THE INVENTION

Hepatocellular carcinoma (HCC) is the third-leading cause of cancer-related deaths globally. Despite improvements in diagnostic and therapeutic approaches, the 5-year survival rate of this cancer is only 7% with a high resistance of HCC to chemotherapy.

Uveal melanomas (UM) represent the most frequent intraocular tumor in adult patients. Up to 50% of the patients will develop metastases, of which 80% die in the first year, and 92% in the first two years. Systemic therapy with alkylating agents, i.e. fotemustine (FM), Dacarbazine (DTIC), or temozolomide (TMZ), have shown only modest efficacy. Consequently, because of the limited efficacy of current treatments, new therapeutic strategies need to be developed.

Hypoxia is a condition in which the body or a region of the body is deprived of adequate oxygen supply. Hypoxia may be classified as either generalized, affecting the whole body, or local, affecting a region of the body. Although hypoxia is often a pathological condition, variations in arterial oxygen concentrations can be part of the normal physiology, for example, during hypoventilation training or strenuous physical exercise. Hypoxia in which there is complete deprivation of oxygen supply is referred to as anoxia.

Hypoxia plays an important and complex role in mediating and regulating the progression of a tumor from a micro-invasive to a metastatic cancer. Unlike normal cells, tumor cells can remain viable in hypoxic environments. HCC is a highly angiogenic cancer containing areas of hypoxia. Hypoxia may promote HCC growth, progression and resistance to ionizing radiation and chemotherapeutic drugs. The on-going development of hypoxic regions in growing tumors provides an opportunity for tumor-selective therapies based on the unique features of hypoxia induced cell responses.

A variety of gene therapy approaches for cancer have failed because it was not possible to achieve effective and specific gene delivery in vivo to the tumors. Selective infection of tumor cells by replication competent viruses, combined with transfer of antitumoral genes is an attractive strategy for cancer therapy. Such an approach may overcome the limitations revealed in clinical trials with replication incompetent vector systems that have shown that efficient therapy requires wide or complete dispersion of the antitumoral gene within the tumor tissue.

### SUMMARY OF THE INVENTION

The present invention provides methods of inducing cell death in a proliferating cell and treating or ameliorating cancer by administering a replication competent virus comprising an antisense molecule against a hypoxia-inducible gene and an anti-cancer therapy. The invention also provides a composition and a kit comprising a replication competent virus comprising one or more antisense molecules against a hypoxia-inducible gene and/or an anti-cancer agent.

According to a first aspect, there is provided a method of inducing cell death in a proliferating cell, the method comprising:
a. contacting said cell with a replication competent retrovirus (RCR) comprising one or more antisense molecules that target at least one hypoxia-inducible gene selected from the group consisting of: HIF-1 and CREB, and
b. exposing the cell to an anti-cancer therapy,

thereby inducing cell death in a proliferating cell.

According to another aspect, there is provided a method of treating, or ameliorating cancer in a subject in need thereof, the method comprising:
a. administering to said subject an RCR comprising one or more antisense molecules that target at least one hypoxia-inducible gene selected from the group consisting of: HIF-1 and CREB, and
b. administering to the subject an anti-cancer therapy,

thereby treating or ameliorating cancer in a subject in need thereof.

According to another aspect, there is provided a pharmaceutical composition comprising a pharmaceutically acceptable carrier and a combination of at least one anti-cancer agent and an RCR comprising one or more antisense molecules that target at least one hypoxia-inducible gene selected from the group consisting of: HIF-1 and CREB.

According to another aspect, there is provided a kit comprising at least one agent selected from:
a. an RCR comprising one or more antisense molecules that target at least one hypoxia-inducible gene selected from the group consisting of: HIF-1 and CREB, the RCR being adapted or identified for co-administration with an anti-cancer agent; and
b. an anti-cancer agent, adapted or identified for co-administration with said RCR.

In some embodiments of the methods, compositions and kits of the invention, said RCR comprises a plurality of antisense molecules that target at least two hypoxia-inducible genes selected from the group consisting of: HIF-1, HIF-2 and CREB. In some embodiments, said RCR comprises a plurality of antisense molecule that target HIF-1, HIF-2 and CREB.

In some embodiments of the methods, compositions and kits of the invention, the RCR comprises a nucleic acid sequence selected from the group consisting of:

(SEQ ID NO: 14)
GAGAGAGGTCCGTCTAATG,
(SEQ ID NO: 15)
CTAACTGGACACAGTGTGTTT,
and
(SEQ ID NO: 16)
CTAACTGGACACAGTGTGTTTAATATATGAAAACACACTGTGTCCAGTTA
GTAAGTCGACTCGCTTATTAAAGTATTCTGATCCGATTATAAAGGATCAG
AATACTTTAATAAGAATGGCGCGTCTTCGAGAGAGGTCCGTCTAATG.

In some embodiments of the methods, compositions and kits of the invention, the retrovirus is a Murine Leukemia virus (MuLV).

In some embodiments, the RCR is administered prior to, or together with, administering the anti-cancer therapy. In some embodiments, administering the RCR potentiates at least one anti-cancer effect of the anti-cancer therapy.

In some embodiments of the methods, compositions of the invention, the anti-cancer therapy comprises a therapy selected from the group consisting of: radiation therapy, chemotherapy, immunotherapy, and any combination thereof. In some embodiments of the kits of the invention the anti-cancer agent is selected from the group consisting of chemotherapy, immunotherapy, and any combination thereof. In some embodiments, the anti-cancer therapy comprises chemotherapy. In some embodiments, the chemotherapy comprises a chemotherapeutic agent selected from the group consisting of: Doxorubicin and Dacarbazine. In some embodiments, the anti-cancer therapy comprises Doxorubicin.

In some embodiments of the methods, compositions and kits of the invention, the proliferating cell is a cancerous cell.

In some embodiments of the methods, compositions and kits of the invention, the cancer is a solid cancer. In some embodiments, the cancer is selected from the group consisting of: hepatoma, melanoma, liver cancer, epithelial cancer, carcinoma and hepatocellular carcinoma. In some embodiments, the cancer is selected from the group consisting of: hepatoma and melanoma. In some embodiments, the cancer is uveal melanoma. In some embodiments, the cancer is hepatocellular carcinoma (HCC).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. A schematic presentation of the various RCRs. A schematic drawing of insertion of an H1 promoter driving the transcription of shRNAS targeting CREB, HIF-1, HIF-2, all three genes and a non-target sequence.

FIGS. 2A-I. Graphs showing the quantification of the efficiency of the knockdown in infected cell lines and the effect on the expression of the targets. (2A-C) Bar graphs depicting levels of mRNA normalized to mRNA in cells infected with vACE-NT (set as 100%). (2D-F) Bar graphs depicting protein levels compared to those of α-tubulin and to levels in cells infected with vACE-NT (set as 100%). (2G) Immunoblot of proteins from infected cells. All reductions were statistically significantly (p<0.05). (2H) Bar graph showing luciferase activity relative to cells infected with vACE-NT (presented as 100%). CRE (dark columns) and HRE (light columns) mediated LUC activity is presented in relative light units (RLU) (P<0.001). (2I) Bar graph depicting VEGF secretion levels in hypoxia relative to normoxia normalized to the ratio in vACE-NT infected cells (p<0.01).

FIGS. 3A-D. Graphs showing the role of CREB, HIF-1, and HIF-2 in protecting HepG2 and FLC4 cells from hypoxia-induced apoptosis and on the response to treatment with DOX. Bar graphs showing HepG2 (3A, 3C) and FLC4 (3B, 3D) infected with each of the viruses. Viability (3A, 3B) and activation of caspase-3 (Cas3) (3C, 3D) were determined every 24 hr. The relative viability and activation of caspase-3 at 72 hour after DOX treatment were normalized to cells infected with each respective RCR in normoxia.

FIGS. 4A-C. Testing the effect of CREB, HIF-1 & 2 on tumor growth in vivo in a mouse xenograft model. (4A) Images of light emission from representative mice taken on days 7 and 35 after injection of HepG2 cells. The same color scale was used for all mice at both time points. (4B) Line graph measuring relative light units (RLU). Measurements were normalized to the readings on day 7 post-injection. (4C) Micrographs of histopathologic analysis of tumors harvested at the end of the experiment. Hematoxylin-eosin staining and the corresponding VEGF immunohistochemistry of tumors are presented (×4 magnification, scale bar 100 μm).

FIG. 5. Immunohistochemical analysis of hypoxia and blood vessels (CD34) of the tumors. Micrographs of tumors infected with RCRs expressing shRNA targeting genes specified on the left column and stained for hypoxia (left column) and for blood vessels (right column). Correlation between the amount of CD34 and hypoxia staining is presented in the top right column for each virus (×10 magnification, scale bar 500 μm).

FIG. 6. Effect of combined treatment on tumor growth monitored by light emission. Micrograph of SCID mice injected with HepG2 cells stably expressing the luc gene and infected with viruses and subsequently injected twice a week with DOX. One mouse from each group is depicted. A scale bar of light emission is provided.

FIGS. 7A-C. Graphs showing the role of CREB, HIF-1, and HIF-2 in protecting HepG2 cells from hypoxia-induced apoptosis and on the response to treatment with DOX. (7A) Line graphs of viability (left panel) and activation of caspase-3 (right panel) in HepG2 cells infected with viruses. The values for each infected cell line were normalized to the readout at time point zero. (7B-C) Line graphs showing viability and activation of caspase 3 in normoxic (7B) and hypoxic (7C) conditions after RCR infection and treatment with 1 mM of DOX.

FIGS. 8A-C. Graphs showing the role of CREB, HIF-1, and HIF-2 in protecting FLC4 cells from hypoxia-induced apoptosis and on the response to treatment with DOX. (8A) Line graphs of viability (left panel) and activation of caspase-3 (right panel) in FLC4 cells infected with viruses. The values for each infected cell line were normalized to the readout at time point zero. (8B-C) Line graphs showing viability and activation of caspase 3 in normoxic (8B) and hypoxic (8C) conditions after RCR infection and treatment with 1 mM of DOX. Hypoxia alone: survival p<0.001, caspase-3 p<0.0001 except for HIF-2 (p=0.9).

FIG. 9. Bar graphs showing viability (left panel) and activation of caspase-3 (right panel) in uveal melanoma cells Mel270 and OMM1 after CREB knockdown and DOX treatment. Expression was normalized to levels in cells infected with vACE-NT.

FIG. 10. Bar graphs showing viability (left panel) and activation of caspase-3 (right panel) in uveal melanoma cells Mel270, OMM2.5 and OMM1 after CREB knockdown and DOX treatment.

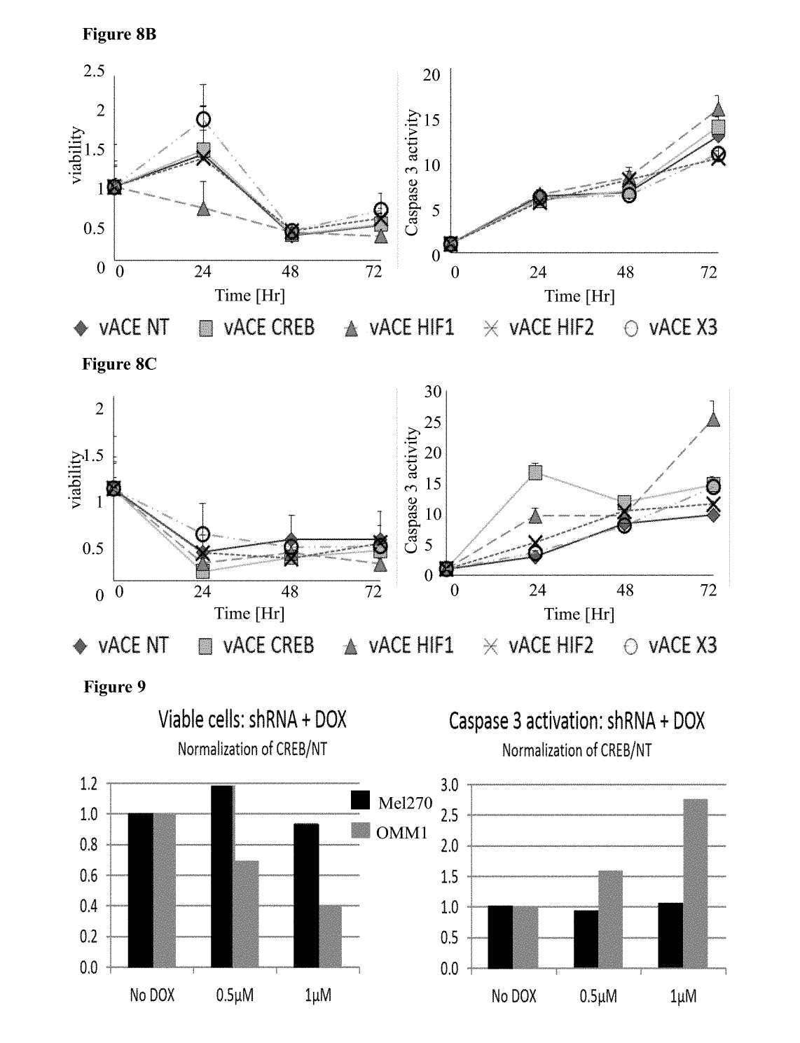

FIG. 11. Bar graphs showing viability and activation of caspase-3 in uveal melanoma cells Mel270, and OMM1 after CREB knockdown and DTIC treatment.

FIG. 12. Bar graphs showing viability and activation of caspase-3 in uveal melanoma cells Mel270, and OMM1 after CREB knockdown and DTIC treatment. Expression was normalized to levels in cells infected with vACE-NT.

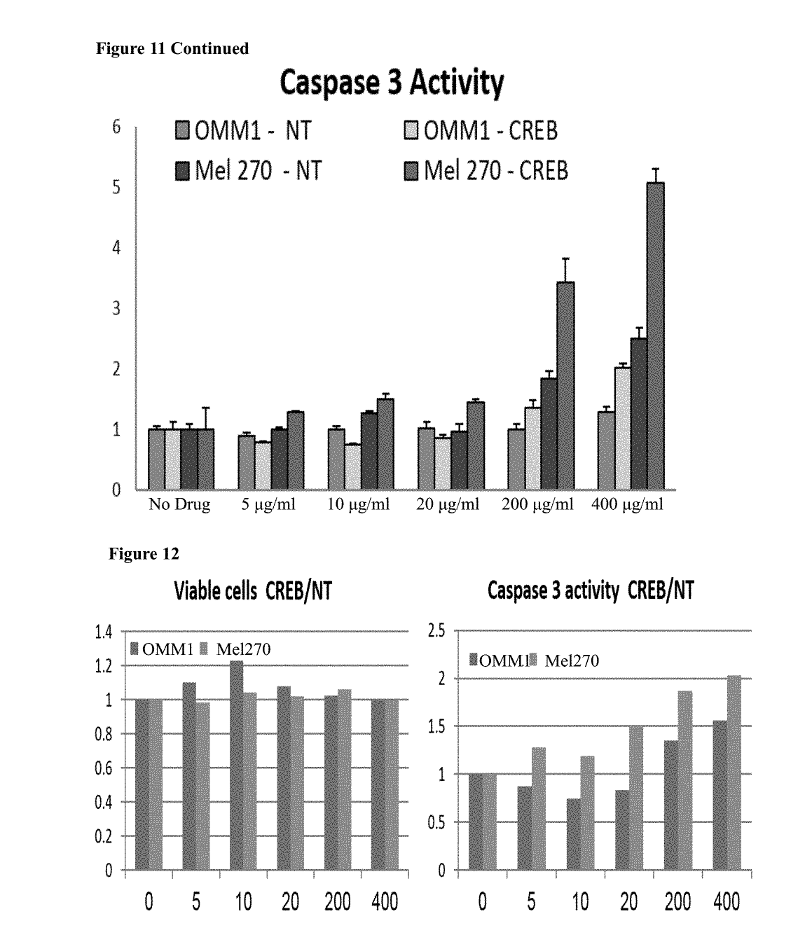

### DETAILED DESCRIPTION OF THE INVENTION

The present invention provides, in some embodiments, methods of inducing cell death in proliferating cells as well as methods of treating cancer, comprising administering a composition comprising a replication competent retrovirus (RCR) comprising one or more antisense molecules that target at least one hypoxia-inducible gene and anti-cancer therapy. There is also provided a composition and a kit comprising the RCR and/or an anti-cancer agent

In some embodiments, the present invention provides a method of inducing cell death in a proliferating cell, the method comprising: (a) contacting the cell with an RCR comprising one or more antisense molecules that target at least one hypoxia-inducible gene selected from the group consisting of: HIF-1 and CREB, and (b) exposing the cell to an anti-cancer therapy, thereby inducing cell death in a proliferating cell.

Hypoxia-Inducible Genes

In some embodiments, the methods, compositions and kits of the invention comprise one or more antisense molecules that target at least one hypoxia-inducible gene. In some embodiments, the at least one hypoxia-inducible gene is selected from the group consisting of: HIF-1 and CREB.

In some embodiments, the methods, compositions and kits of the invention comprise one or more antisense molecules that target at HIF-1 and CREB and optionally at least one additional hypoxia-inducible gene such as HIF-2. In some embodiments, the at least one hypoxia-inducible gene is selected from the group consisting of: HIF-1, CREB, and HIF-2.

HIF-1, as used herein, refers to the gene HIF1A. In some embodiments, HIF-1 comprises the nucleic acid sequence of NCBI Reference Sequence: NM_001530.3 (SEQ ID NO: 18). In some embodiments, HIF-1 comprises the nucleic acid sequence of NCBI Reference Sequence: NM_181054.2 (SEQ ID NO: 19). In some embodiments, HIF-1 comprises the nucleic acid sequence of NCBI Reference Sequence: NM_001243084.1 (SEQ ID NO: 20).

HIF-2, as used herein, refers to the gene EPAS1. In some embodiments, HIF-2 comprises the nucleic acid sequence of NCBI Reference Sequence: NM 001430.4 (SEQ ID NO: 21).

CREB, as used herein, refers to the gene CREB1. In some embodiments, CREB comprises the nucleic acid sequence of NCBI Reference Sequence: NM_134442.4 (SEQ ID NO: 22). In some embodiments, CREB comprises the nucleic acid sequence of NCBI Reference Sequence: NM_004379.4 (SEQ ID NO: 23). In some embodiments, CREB comprises the nucleic acid sequence of NCBI Reference Sequence: NM_001320793.1 (SEQ ID NO: 24).

In some embodiments, the antisense molecule that targets CREB comprises or consists of the sequence GAGAGAGGTCCGTCTAATG (SEQ ID NO: 14).

In some embodiments, the antisense molecule that targets HIF-1 comprises or consists of the sequence CTAACTGGACACAGTGTGTTT (SEQ ID NO: 15).

In some embodiments, the antisense molecule that targets HIF-2 comprises or consists of the sequence CTTATTAAAGTATTCTGATCC (SEQ ID NO: 17).

As used herein, the term “antisense molecule” refers to a nucleic acid molecule whose sequence is a reverse complement to the sequence found in the mRNA of a specific gene. The gene to whose mRNA the molecule is reverse complementary is the “target” of the antisense molecule. In some embodiments, the antisense molecule, may have 0, 1, 2, 3 or more mismatches with its target's mRNA. Each possibility represents a different embodiment of the invention.

It will be well understood to one skilled in the art that targeting a gene constitutes targeting of the mRNA transcribed from the gene. Thus, the antisense molecule is substantially a reverse complement to the mRNA of a target gene. It will further be well understood, that binding of the antisense molecule to its target will result in reduction in the protein produced by that mRNA. This may be due to destruction of the mRNA or due to poor translation of the mRNA.

In some embodiments, the antisense molecule reduces protein produced by an mRNA by at least 20%, 25%, 30%, 35%, 40%, 45%, 50%, 55%, 60%, 65%, 70%, 75%, 80%, 85%, 90%, 95%, or 99%. Each possibility represents a different embodiment of the invention.

In some embodiments, the antisense molecule is an RNA interference (RNAi) molecule. In some embodiment, the antisense molecule is selected from a group consisting of: a small interfering RNA (siRNA) and a short hairpin RNA (shRNA). In some embodiments, the antisense molecule is a shRNA.

It will be understood to one skilled in the art that siRNAs and shRNAs against specific targets can be determined or found using well known programs and websites, such as, for example, siRNA finder websites supplied by commercial nucleotide producers (ThermoFisher, Ambion, InvivoGen), siRNA design websites (http://www.rnaiweb.com), and listings of validated siRNAs/shRNAs (web.mit.edu/sirna/browse-main.html). Conversion from siRNA to shRNA (or vice versa) will also be well understood to one skilled in the art, and websites and programs for doing so are well known. Non-limiting examples of such websites and programs include, http://www.rnaiweb.com and rnaidesigner.thermofisher.com/rnaiexpress/help/convert_sirna_to_shrna.htm.

In another embodiment, the present invention provides an RCR comprising at least one, at least two or three antisense (e.g., shRNA) molecules selected from the group comprising: HIF-1, HIF-2 and CREB. In some embodiments, the antisense molecules are located concomitantly, simultaneously, sequentially, consecutively, in tandem or separately on the RCR. In some embodiments, there is a plurality of each identical anti-sense molecule on the RCR. In another embodiment, there is provided a plurality of RCR, each RCR comprises at least one antisense molecules (e.g., shRNA) targeting at least one hypoxia-inducible gene (e.g., HIF-1, HIF-2 and CREB).

In some embodiments, the RCR disclosed herein comprises one or more antisense molecules targeting CREB. In some embodiments, the RCR disclosed herein comprises one or more antisense molecules comprising a nucleic acid sequence complementary to at least 5 contiguous bases derived from CREB. In some embodiments, the RCR disclosed herein comprises one or more antisense molecules targeting HIF-1. In some embodiments, the RCR disclosed herein comprises one or more antisense molecules comprising a nucleic acid sequence complementary to at least 5 contiguous bases derived from HIF-1 In some embodiments, the RCR disclosed herein comprises one or more antisense molecules targeting HIF-2. In some embodiments, the RCR disclosed herein comprises one or more antisense molecules comprising a nucleic acid sequence complementary to at least 5 contiguous bases derived from HIF-2.

It will be understood by one skilled in the art that a combination of at least one, but optionally more antisense molecules can be employed to target any of the genes in any of the RCRs of the invention.

The term “replication competent retrovirus” or “RCR” as used herein, refers to a virus that can only infect a cell that divides. The requirement for cellular division, means that an RCR cannot infect quiescent and non-dividing cells, such as make up most of the cells of a living organism. However, an RCR can be passed laterally from one proliferating cell to the next, such as might be found in a tumor. One non-limiting example of a RCR is a virus that lacks a nuclear localization signal for active transport across an intact nuclear membrane. In some embodiments, the RCR is a Murine Leukemia virus (MuLV).

In one embodiment, the RCR comprises a HIF-1-shRNA and a HIF-2-shRNA. In one embodiment, the RCR comprises a HIF-1-shRNA, and CREB-shRNA. In one embodiment, the RCR comprises a HIF-1-shRNA, a HIF-2-shRNA and a CREB-shRNA. In some embodiments, the RCR comprises the sequence

(SEQ ID NO: 16)
CTAACTGGACACAGTGTGTTTAATATATGAAAACACACTGTGTCCAGTTA
GTAAGTCGACTCGCTTATTAAAGTATTCTGATCCGATTATAAAGGATCAG
AATACTTTAATAAGAATGGCGCGTCTTCGAGAGAGGTCCGTCTAATG.

The DNA sequence encoding CREB-shRNA, in some embodiments, comprises or consists of the nucleic acid sequence:

(pACE-CREB, SEQ ID NO: 1)
GAGAGAGGTCCGTCTAATGTTCAAGAGACATTAGACGGACCTCTCTCTTT
TT.

The DNA sequence encoding HIF1-shRNA, in some embodiments, comprises or consists of the nucleic acid sequence:

(pACE-HIF1, SEQ ID NO: 2)
CTAACTGGACACAGTGTGTTTAATATATGAAAACACACTGTGTCCAGTTA
GTTTTTT.

The DNA sequence encoding HIF2-shRNA, in some embodiments, comprises or consists of the nucleic acid sequence:

(pACE-HIF2, SEQ ID NO: 3)
ATTAAAGTATTCTGATCCGA.TTATAAAGGATCAGAATACTTTAATAAGT
TTTTTT.

The DNA sequence combining at least 3 shRNAs, in some embodiments, comprises or consists of the nucleic acid sequence: CTAACTGGACACAGTGTGTTTAATATATGAAAACACACTGTGTCCAGTTAGTA AGTCGACTCGCTTATTAAAGTATTCTGATCCGATTATAAAGGATCAGAATACTT TAATAAGAATGGCGCGTCTTCGAGAGAGGTCCGTCTAATGCCTGAACCACATT AGACGGACCTCTCTCTTTTTT (pACE X3, SEQ ID NO: 4). This vector includes shRNAs targeting CREB, HIF-1 and HIF-2.

In another embodiment, the DNA sequence encoding an antisense of the invention is at least 70% homologous or identical to any sequence of SEQ ID Nos: 1-4, 14-16. In another embodiment, the DNA sequence encoding an antisense of the invention is at least 75% homologous or identical to any sequence of SEQ ID Nos: 1-4, 14-16. In another embodiment, of the invention is at least 80% homologous or identical to any sequence of SEQ ID Nos: 1-4, 14-16. In another embodiment, the DNA sequence encoding an antisense of the invention is at least 85% homologous or identical to any sequence of SEQ ID Nos: 1-4, 14-16. In another embodiment, the DNA sequence encoding an antisense of the invention is at least 90% homologous or identical to any sequence of SEQ ID Nos: 1-4, 14-16. In another embodiment, the DNA sequence encoding an antisense of the invention is at least 92% homologous or identical to any sequence of SEQ ID Nos: 1-4, 14-16. In another embodiment, the DNA sequence encoding an antisense of the invention is at least 95% homologous or identical to any sequence of SEQ ID Nos: 1-4, 14-16. In another embodiment, the DNA sequence encoding an antisense of the invention is at least 98% homologous or identical to any sequence of SEQ ID Nos: 1-4, 14-16.

In some embodiments, the antisense molecule is part of a vector that is introduced into the virus. In some embodiments, the vector is the pACE vector. In some embodiments, the vector is a modified pACE vector.

In one embodiment, various methods can be used to introduce the expression vector of the present invention into cells or a virus. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors. A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods. In some embodiments, introduction of nucleic acid by viral infection offers several advantages over other methods such as lipofection and electroporation, since higher transfection efficiency and/or cell specificity can be obtained due to the infectious nature of viruses.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid” which refers to a circular double stranded DNA loop into which additional DNA segments can be inserted, such as by standard molecular cloning techniques. Another type of vector, wherein virally-derived DNA or RNA sequences are present in the virus (e.g. retroviruses, replication defective retroviruses, adenoviruses, replication defective adenoviruses, and adeno-associated viruses). Viral vectors also include polynucleotides carried by a virus for transfecting into host cells. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. Common expression vectors of utility in recombinant DNA techniques are often in the form of plasmids.

Recombinant expression vectors can comprise a nucleic acid coding for the protein of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into cells or a virus by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), and/or the like.

General methods in molecular and cellular biochemistry, such as may be useful for carrying out DNA and protein recombination, as well as other techniques described herein, can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

In some embodiments, expression of the antisense molecules of the invention are driven by an operably linked tissue-specific promoter. Tissue-specific promoters are only active in those tissues, and will restrict expression of the antisense molecules to those tissues. Thus, even in the viruses of the invention infect proliferating non-cancer cells, the antisense (e.g. shRNAs) molecule of the invention will not be expressed. In some embodiments, expression of the antisense molecules of the invention are driven by an operably linking cancer-specific promoter.

Tissue and cancer specific promoters are well known in the art. Some non-limiting examples of tissue and cell-specific promoters include: CD45 promoter—hematopoietic cells, B29 promoter—B cells, CD68 promoter—macrophages, Desmin promoter—muscle, elastase 1 promoter—pancreas, GFAP promoter—astrocytes, SP-B promoter—lung, SYN1 promoter—neurons, and SV40/bAlb promoter—Liver.

Some non-limiting examples of cancer—specific promoter include: AFP promoter—hepatocellular carcinoma, CCKAR promoter—pancreatic cancer, CEA promoter—epithelial cancers, COX2 promoter—solid tumors, and MUC1 promoter—carcinoma cells.

### Anti-Cancer Therapy

An RCR comprising at least one or at least two antisense molecules (e.g., shRNAs) selected from the group comprising: a HIF-1, and CREB, is used in some embodiments, to sensitize proliferating cells and/or cancerous cells to anti-cancer therapy.

“Anti-cancer therapy”, as used herein, refers to a drug, RNA, gene therapy or other treatment, such as irradiation, that slows, ameliorates, halts or abolishes growth of a cancer cell, or alternatively kills a cancer cell. In some embodiments, the anti-cancer therapy includes an anti-cancer agent such as, but not limited to, a chemotherapeutic agent. A chemotherapeutic agent or an anti-cancer therapy, in some embodiments, is an agent or a treatment which impairs a cellular hypoxia response. A chemotherapeutic agent, in some embodiments, is an alkylating agent.

Chemotherapeutic agents will be well known to one skilled in the art, but a non-limiting list includes: cyclophosphamide, mechlorethamine, chlorambucil, melphalan, doxorubicin, dacarbazine, nitrosoureas, temozolomide, daunorubicin, epirubicin, idarubicin, mitoxantrone, valrubicin, paclitaxel, docetaxel, abraxane, taxotere, varinostat, romidepsin, irinotecan, topotecan, etoposide, teniposide, tafluposide, bortezomib, erlotinib, getitinib, imatinib, vermurafenib, vismodegib, azacytidine, azathioprine, capecitabine, cytarabine, doxifluridine, fluorouracil, gemcitabine, hydroxyurea, mercaptopurine, methotrexate, tioguanine, bleomycin, actinomycin, carboplatin, cisplatin, oxaliplatin, tretinoin, alitretinoin, bexarotene, vinblastine, vincristine, vindesine, and vinorelbine.

In some embodiments, the anti-cancer therapy comprises a therapy selected from the group consisting of: radiation therapy, chemotherapy, immunotherapy, and any combination thereof.

In some embodiments, the anti-cancer therapy comprises a therapy selected from the group consisting of: radiation therapy, chemotherapy, immunotherapy, hormone therapy, antibody therapy, a signal transduction inhibitor, a gene expression modulator, an apoptosis inducer, an angiogenesis inhibitor, an inhibitor of cellular hypoxia response, and any combination thereof.

In some embodiments, an inhibitor of cellular hypoxia response is a drug. In some embodiments, chemotherapy is an inhibitor of cellular hypoxia response. In some embodiments, an inhibitor of cellular hypoxia response is Doxorubicin. In some embodiments, an inhibitor of cellular hypoxia response is Dacarbazine. An inhibitor of cellular hypoxia response, in one embodiment, is an antineoplastic agent. An inhibitor of cellular hypoxia response, in one embodiment, is a cytotoxic agent. An inhibitor of cellular hypoxia response, in one embodiment, is an alkylating agent. An inhibitor of cellular hypoxia response, in one embodiment, is a chemotherapeutic agent. An inhibitor of cellular hypoxia response, in one embodiment, is a cancer chemotherapeutic agent. An inhibitor of cellular hypoxia response, in one embodiment, is a tumor chemotherapeutic agent.

In some embodiments, chemotherapy comprises a chemotherapeutic agent selected from the group consisting of: Doxorubicin and Dacarbazine. In some embodiments, chemotherapy comprises Doxorubicin. In some embodiments, chemotherapy comprises Dacarbazine.

### Method of Use

In some embodiments, the RCR is administered together with the anti-cancer therapy to a proliferating cell. In some embodiments, the RCR and the anti-cancer therapy are in a single composition. In some embodiments, the RCR is administered separately from the anti-cancer therapy. In some embodiments, the RCR is administered before the anti-cancer therapy is applied or administered to a proliferating cell. In some embodiments, the RCR is administered at the same time as the anti-cancer therapy. In some embodiments, the RCR is administered after the anti-cancer therapy.

In another embodiment, the RCR and the anti-cancer therapy are used concomitantly, simultaneously, sequentially, consecutively, separately or combined. In another embodiment, the present invention provides use of at least two compositions concomitantly, simultaneously, sequentially, consecutively, separately or combined, wherein a first composition comprises at least one, at least two or three shRNA molecules selected from the group comprising: HIF-1-shRNA, and CREB-shRNA and a second composition comprising at least one anti-cancer therapy, for inhibiting proliferation of a cell undergoing mitosis and/or inducing cell death in a cell undergoing mitosis.

In some embodiments, the proliferating cell is a cell of a cell line. In some embodiments, the proliferating cell is a cell in culture. In some embodiments, the proliferating cell is a cancerous cell. In some embodiments, the proliferating cell is in vitro. In some embodiments, the proliferating cell is in vivo. In some embodiments, the proliferating cell is within a subject. In some embodiments, that subject is a human.

By another aspect, the present invention concerns a pharmaceutical composition comprising a pharmaceutically acceptable carrier and a combination of at least one anti-cancer agent and an RCR as described above.

The pharmaceutical composition is, in some embodiments, formulated for administration to a subject suffering from cancer. In some embodiments, the composition is formulated for parenteral administration. In some embodiments, the composition is formulated for intraperitoneal administration. In some embodiments, the composition is formulated for direct injection into a tumor. In some embodiments, the composition is formulated for direct injection adjacent to the tumor. In some embodiments, the composition is formulated for ocular administration, including direct ocular administration by injection. In some embodiments, the composition is formulated for oral administration.

Suitable routes of administration may, for example, include oral, rectal, transmucosal, transnasal, intestinal or parenteral delivery, including intramuscular, subcutaneous and intramedullary injections as well as intrathecal, direct intraventricular, intravenous, inrtaperitoneal, intranasal, intraarterial, intravesicle (into the bladder) or intraocular injections. In some embodiments, the pharmaceutical composition comprising the RCR and/or the anti-cancer agent is administered intra-tumorally. In one embodiment, one may administer the pharmaceutical composition comprising the RCR and/or the anti-cancer agent in a local rather than systemic manner, for example, via injection of directly into a tissue region of a patient. Techniques for formulation and administration of drugs may be found in “Remington's Pharmaceutical Sciences,” Mack Publishing Co., Easton, Pa., latest edition, which is incorporated herein by reference.

A composition such as described herein is used, in some embodiments, for treating cancer. A composition such as described herein is used, in some embodiments, for inhibiting the growth of a tumor. A composition such as described herein is used, in some embodiments, for inhibiting the proliferation of a cell. A composition such as described herein is used, in some embodiments, for sensitizing a cancerous cell and/or a tumor cell to an anti-cancer therapy. A composition such as described herein is used, in some embodiments, for sensitizing a proliferating cell to an anti-proliferation agent or therapy. A composition such as described herein is used, in some embodiments, for treating a disease characterized by aberrant cell proliferation. A composition such as described herein is used, in some embodiments, for inhibiting angiogenesis. A composition such as described herein is used, in some embodiments, for inducing caspase-3 expression. A composition such as described herein is used, in some embodiments, for inhibiting VEGF expression.

### Cancer Treatment

By another aspect, there is provided a method of treating, or ameliorating cancer in a subject in need thereof, the method comprising: (a) administering to the subject a replication competent retrovirus (RCR) comprising an antisense molecule that targets a hypoxia-inducible gene selected from the group consisting of: HIF-1 and CREB, and (b) administering to the subject an anti-cancer therapy, thereby treating or ameliorating cancer in a subject in need thereof.

In some embodiments, the cancer is a solid cancer. In some embodiments, the cancer comprises regions of hypoxia. In one embodiment, cancer is liver cancer. In one embodiment, cancer is carcinoma. In one embodiment, cancer is epithelial cancer. In one embodiment, cancer is hepatocellular carcinoma. In one embodiment, cancer is epithelial cancer. In one embodiment, cancer is hepatoma. In one embodiment, cancer is uveal melanoma. In one embodiment, cancer is melanoma. In some embodiments, the cancer is selected from the group consisting of: hepatoma, melanoma, liver cancer, epithelial cancer, carcinoma and hepatocellular carcinoma.

It will be well understood to one skilled in the art, that the method of treating cancer provided herein will be effective in any cancer that contains a hypoxic microenvironment. By its nature, a solid tumor has an interior which is frequently oxygen starved. As such, in some embodiments the methods of the current invention are practiced on all solid cancers, included but not limited to: retinoblastoma, colon cancer, breast cancer, cutaneous melanoma, and pancreatic cancer.

Further, as the methods described herein have been shown to inhibit VEGF expressions, cancers that express VEGF would also be susceptible. Examples of such cancers include, but are not limited to: breast cancer, non-small cell lung cancer, squamous cell lung cancer, and colorectal cancer. Additionally, cancers of the blood such as leukemias and lymphomas, are frequently VEGF dependent and therefore the methods of treating cancer presented herein may be effective in these and other VEGF expression non-solid cancers. In some embodiments, the cancer is selected from the group of VEGF expression cancers consisting of: breast cancer, non-small cell lung cancer, squamous cell lung cancer, and colorectal cancer. In some embodiments, the cancer is a VEGF expressing non-solid cancer. In some embodiments, the cancer is a leukemia or a lymphoma.

In some embodiments, the treating or ameliorating cancer comprises enhancing the activity or the efficacy of the anti-cancer therapy, and wherein the enhancing is selected from the group consisting of: reducing the toxicity of the anti-cancer therapy, reducing the dose of the anti-cancer therapy, reducing a side effect associated with the anti-cancer therapy, and a combination thereof.

In some embodiments, the toxicity is reduced by at least 10%, 20%, 30%, 40%, 50%, 60%, 70%, 80%, 90%, 99%, or 100%. Each possibility represents a separate embodiment of the present invention.

In some embodiments, the dose is reduced by at least 10%, 20%, 30%, 40%, 50%, 60%, 70%, 80%, 90%, or 99%. Each possibility represents a separate embodiment of the present invention.

In some embodiments, a side effect is reduced by at least 10%, 20%, 30%, 40%, 50%, 60%, 70%, 80%, 90%, or 99%. Each possibility represents a separate embodiment of the present invention.

In some embodiments, treating or ameliorating the cancer comprises sensitizing a cancer cell to an anti-cancer therapy. The term “sensitizing” as used herein comprises: reducing LD50 of an anti-cancer therapy, reducing the effective amount of an anti-cancer therapy, reducing side-effects associated with an anti-cancer therapy, reducing the toxicity of an anti-cancer therapy, improving the efficiency of treatment by anti-cancer therapy, inducing caspase-3 expression, inhibiting VEGF expression, or any combination thereof.

In some embodiments, there is provided a method of potentiating the anti-cancer effect of an anti-cancer therapy, the method comprising: administering to a subject undergoing said anti-cancer therapy an effective amount of an RCR comprising an antisense molecule that targets at least one hypoxia-inducible gene selected from the group consisting of: HIF-1 and CREB.

In some embodiments, administering said RCR potentiates at least one anti-cancer effect of the anti-cancer therapy. In some embodiments, the RCR is administered before the anti-cancer therapy and potentiates the therapy. In some embodiments, the RCR is administered with the anti-cancer therapy and potentiates the therapy. In some embodiments, the potentiation enhances the anti-cancer therapy by at least 10%, 20%, 30%, 40%, 0.50%, 60%, 70%, 80%, 90%, or 99%. Each possibility represents a separate embodiment of the present invention.

In some embodiments, the anti-cancer effect is selected from the group consisting of: reducing tumor cell proliferation, enhancing tumor cell apoptosis, enhancing immune cell killing of tumor cells, reducing tumor cell invasion, reducing tumor cell metastasis, and a combination thereof.

In one embodiment, the present invention provides a method for reducing the effective dose of anti-cancer therapy, such as DOX or DTIC, to a subject in need thereof, the method comprising treating the subject with an RCR comprising at least one or at least two or three antisense molecules that target a gene selected from the group comprising or consisting: HIF-1, CREB, and HIF-2.

In one embodiment, the present invention provides a method for reducing a side effect associated with an anti-cancer therapy comprising treating the subject with an RCR comprising at least one or at least two or three antisense molecules that target a gene selected from the group comprising or consisting: HIF-1, CREB and HIF-2.

In some embodiments, reducing a side effect is improving efficacy and/or improving safety profile and/or decreasing side effects of an anti-cancer therapy.

In one embodiment, the present invention provides a method for sensitizing a cancerous cell, a proliferating cell and/or a tumor cell to DOX or DTIC comprising: contacting the cancerous cell and/or the tumor cell with an RCR comprising at least one or at least two or three antisense molecules that target a gene selected from the group comprising or consisting: HIF-1, CREB, and HIF-2. In one embodiment, a method for sensitizing includes sensitizing for an anti-cancer therapy such as but not limited to a chemotherapeutic agent, an agent which impairs a cellular hypoxia response, and/or an alkylating agent. In one embodiment, a method for sensitizing includes sensitizing for an alkylating agent.

In one embodiment, the present invention provides a method for reducing and/or inhibiting the expression of VEGF in a cancer cell or a tumor, comprising contacting the tumor with a composition as described herein.

In one embodiment, the present invention provides a method for inhibiting angiogenesis in a tumor or in the proximity of a cancerous tissue, comprising contacting the tumor and/or the cancerous tissue with a composition as described herein. In one embodiment, the present invention provides a method for inhibiting angiogenesis and/or vascularization in a tissue, comprising contacting the tissue with a composition as described herein. In one embodiment, the tissue is a cancerous tissue. In one embodiment, the tissue is a tumor. In one embodiment, the tissue is a solid tumor. In one embodiment, the tissue is afflicted with carcinoma.

In some embodiments, the methods of the invention are performed after an anti-cancer therapy has lost effectiveness or is completely ineffective. In some embodiments, the anti-cancer therapy of the invention is the anti-cancer therapy that is no longer effective. In some embodiments, the methods of the invention can be considered a second-line therapy. In some embodiments, the RCR potentiates the anti-cancer therapy and thus is a second-line therapy.

### Kits and Compositions

By another aspect, there is provided a kit or a composition comprising a replication competent retrovirus (RCR) comprising at least one antisense molecule that targets a hypoxia-inducible gene selected from the group consisting of: HIF-1 and CREB, the RCR or composition comprising same is adapted or identified for co-administration with an anti-cancer agent.

By another aspect, there is provided a kit or a composition comprising an anti-cancer agent, adapted or identified for co-administration with an RCR comprising at least one antisense molecule that targets a hypoxia-inducible gene selected from the group consisting of: HIF-1 and CREB.

By another aspect, there is provided a kit or a composition comprising: (a) an RCR comprising at least one antisense molecule that targets a hypoxia-inducible gene selected from the group consisting of: HIF-1 and CREB; and (b) an anti-cancer agent. In some embodiments, the RCR is adapted or identified for co-administration with the anti-cancer agent.

In some embodiments, the anti-cancer agent is selected from the group comprising: chemotherapy, cellular hypoxia impairment response agent, a hormone therapy, signal transduction inhibitor, gene expression modulator, apoptosis inducer, angiogenesis inhibitor, immunotherapy, antibody therapy, or any combination thereof. In some embodiments, the anti-cancer therapy is selected from the group consisting of: Doxorubicin and Dacarbazine. In some embodiments, the anti-cancer therapy comprises Doxorubicin.

In one embodiment, the present invention provides two compositions: a first composition comprising an RCR adapted for co-administration with an anti-cancer therapy, and comprising an antisense molecule as described herein; and a second composition comprising at least one anti-cancer therapy as described herein.

In one embodiment, the present invention provides a kit for treating a subject afflicted with a solid cancer. In one embodiment, the present invention provides a kit for treating a subject afflicted with a cancer selected from the group consisting of: hepatoma, melanoma, liver cancer, epithelial cancer, carcinoma and hepatocellular carcinoma. In one embodiment, the present invention provides a kit for treating a subject afflicted with uveal melanoma.

In one embodiment, the present invention provides a kit comprising: (a) a replication competent retrovirus (RCR) adapted for co-administration with an anti-cancer agent, comprising at least one of: a HIF-1-shRNA, a HIF-2-shRNA or a CREB-shRNA; and (b) DOX and/or DTIC.

The term “adapted for co-administration with an anti-cancer therapy” as used herein, refers to the virus being present in a form such that it can be safely and easily administered to a subject. Co-administration, in some non-limiting embodiments, can be done orally, by injection, or by inhalation. In some embodiments, the adapted virus will be comprised within a pharmaceutical composition such as can be safely and easily administered to a subject. In some embodiments, the pharmaceutical composition comprises the virus and a pharmaceutically acceptable carrier or excipient.

The term “identified for co-administration” refers to the fact that the agent appears with a label, and has received regulatory approval, to be administered in combination with the other agent. As will be appreciated to a skilled artisan, a first agent identified for co-administration with a second agent, may be sold and/or packaged separately or in combination with the second agent.

### Pharmaceutical Composition

As used herein, the term “carrier,” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic, pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference. Other general references are provided throughout this document.

### Materials and Methods

### Cell Culture

Human HCC HepG2 cell line (verified by STR analysis) was grown in Dulbecco's modified Eagle's medium (DMEM) supplemented with 10% fetal bovine serum, 2 mM glutamine, 100 IU/ml penicillin, 100 μg/ml streptomycin (Biological Industries) and incubated at 37° C. in a humidified atmosphere with 5% CO₂. To culture cells in hypoxia the cells were incubated in hypoxia jars at 0.5% 02 (AnaeroGen, Oxoid).

### Plasmids and Viruses

The plasmid pACE-GFP contained a full-length replication-competent amphotropic MuLV provirus with an additional internal ribosome entry site (IRES)-GFP cassette flanked with BsiWI and Not1 restriction enzymes sites. This cassette was replaced by oligonucleotides harboring the H1 promoter driving the transcription of one of the following shRNA sequences:

(pACE-CREB, SEQ ID NO: 1)
5′_GAGAGAGGTCCGTCTAATGTTCAAGAGACATTAGACGGACCTCTCT
CTTTTT.
(pACE-HIF1, SEQ ID NO: 2)
5′_CTAACTGGACACAGTGTGTTTAATATATGAAAACACACTGTGTCCA
GTTAGTTTTTT.
(pACE-HIF2, SEQ ID NO: 3)
5′_ATTAAAGTATTCTGATCCGATTATAAAGGATCAGAATACTTTAATA
AGTTTTTT.

5′_CTAACTGGACACAGTGTGTTTAATATATGAAAACACACTGTGTCCA GTTAGTAAGTCGACTCGCTTATTAAAGTATTCTGATCCGATTATAAAGGATCAG AATACTTTAATAAGAATGGCGCGTCTTCGAGAGAGGTCCGTCTAATGCCTGAA CCACATTAGACGGACCTCTCTCTTTTTT (pACE X3, SEQ ID NO: 4). This vector included shRNA targeting CREB, HIF-1 and HIF-2.

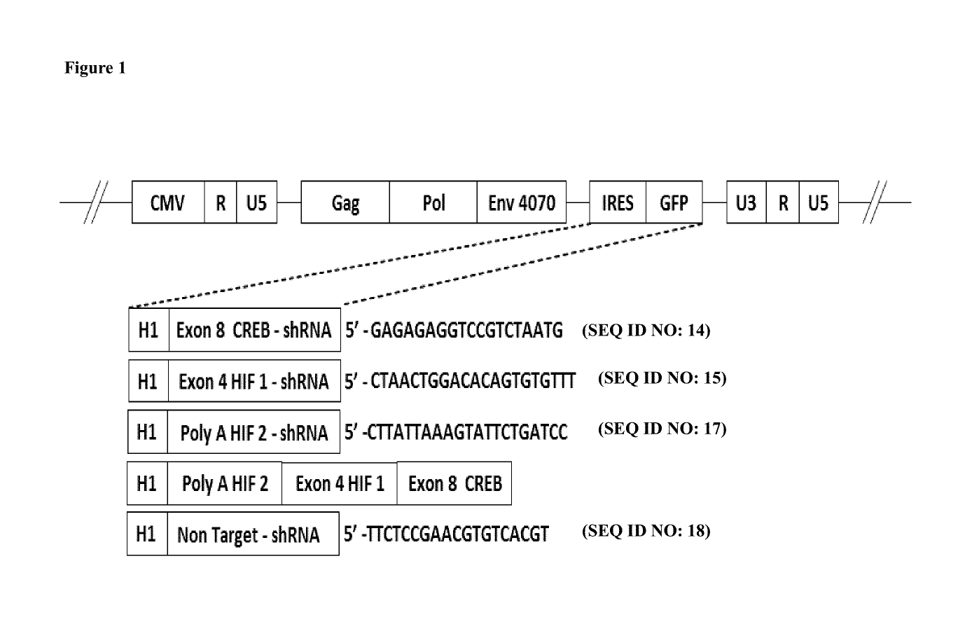

5′_ACCAAGATGAAGAGCACCAACCTGAACCATTGGTGCTCTTCATCTT GGTTTTTTT (pACE-NT. Non-target shRNA, SEQ ID NO: 5). See FIG. 1 for a schematic presentation of the vectors.

### Virus Production

HEK293T cells were transiently transfected with either one of the plasmids, described above and in FIG. 1, using FuGENE HD (Promega). The medium was harvested 48 h later, filtered (MILLEX-HV, PVDF 0.45μ) and stored at −80° C.

### Western Blot Analysis

Equal amounts of total protein were prepared in Laemmli SDS loading buffer, resolved on 10% SDS-PAGE and transferred to PVDF membranes (Millipore). For detection of CREB, HIF-1 or HIF-2, membranes were blocked for half an hour in TBS-T (20 mM Tris pH 7.4, 150 mM NaCl, 0.1% Tween-20) containing 5% skim milk (Difco) and incubated overnight (4° C.) with either CREB (Santa Cruz), HIF-1 (Abcam), HIF 2 (Novus Biologicals) or GAPDH (Santa Cruz) primary antibodies. The blots were washed, incubated with secondary HRP-conjugated antibody (Promega) for one hour, then washed again and were visualized using the enhanced chemiluminescence (ECL) system (Promega). Blots were scanned by the MiniBIS Pro (DNR), and band intensities were quantified by the TINA 20 program (Raytest).

### Quantitative Real-Time PCR

RNA was extracted from the cells using the SV Total RNA isolation System (Promega), according to the manufacturer's instructions. The purified RNA samples were subjected to reverse transcription using GoScript (Promega), monitored by quantitative 7900HT real-time PCR apparatus (Applied Biosystems) utilizing the GoTaq Real-Time PCR reagents (Promega) and the specific primers: CREB: fp-5′_CCCAGCACTTCCTACACAGCCTGC, (SEQ ID NO: 6) rp5′_CGAGCTGCTTCCTGTTCTTCATTAGACG, (SEQ ID NO: 7) HIF-1: fp5′_GGGATTAACTCAGTTGAACTAACTGG, (SEQ ID NO: 8) rp5′_CCTTTTTCACAAGGCCATTTCTGTGTG (SEQ ID NO: 9), HIF-2: fp5′_ACAAGGTGTCAG-GCATGGCAAGC (SEQ ID NO: 10) rp5′_CGTTCACCTCACAGTCATATCTGG (SEQ ID NO: 11). The results were normalized to the cellular house-keeping gene GAPDH: fp-5′CCATCTTCCAGGAGCGAGATCC (SEQ ID NO: 12), rp-5′_GCAAATGAGCCCCAGCTTCTCC (SEQ ID NO: 13).

### Luciferase Assay

HepG2 cells were infected with vACE HIF1, vACE HIF2, vACE CREB, vACE X3 or vACE NT and seeded in 6-well plates at a concentration of 500,000 cells/well for 24 hours. The infected cells were co-transfected (3 μg DNA) with either the CRE mediated luciferase (luc) reporter plasmid vector, pCREluc or by the ERE controlled luc gene reporter plasmid (Meyuhas R, et al., 2008, Molecular Cancer Research, 6:1397-409) together with 0.25 μg of an expression vector expressing the Renilla luciferase gene, phRLSV40, as a transfection control (Promega) using FuGENE HD (Promega). Luciferase activity was determined 48 h post transfection, according to the manufacturer's instructions (Dual Luciferase reporter assay system, Promega) by an automatic Mithras LB 940 photoluminometer (Berthold Technologies). The results were normalized to Renilla luciferase activity.

### VEGF Expression Assay

Infected cells with either one of the viruses mentioned above were seeded at a concentration of 3000 cells/well in 96-well plates and incubated at normoxic and hypoxic conditions for 24 hours (2-4 repeats). The supernatant vascular endothelial growth factor (VEGF) levels were quantified by enzyme-linked immunosorbent assays (ELISA) following the manufacturers' instructions (R&D Systems). VEGF levels were normalized to the cell count and to the VEGF levels from cells infected with vACE-NT.

### Cell Viability and Activation of Caspase-3

Infected cells with one of the recombinant viruses mentioned above were seeded at a concentration of 3000 cells/well in 96-well plates (6 repeats) and incubated at normoxic and hypoxic conditions up to 72 h. At the indicated time points cells viability and Caspase-3 activity was determined by the Fluorescent Cell Viability and Caspase-Glo 3/7 Assays (Promega) according to the manufacturer's instructions.

### Xenograft Mouse Model

The animal protocol used in this study was approved by the IACUC of the Hebrew University of Jerusalem. HepG2 cells infected with one of the recombinant viruses were harvested and 4×10⁶ cells in 100 μl were injected subcutaneously (SC) above the foreleg of severe combined immunodeficiency (SCID) mice weighing 20-24 gr. Tumor growth was monitored following intraperitoneal (IP) injection of D-Luciferin 300 mg/0.1cc/mouse (Promega) 10 minutes before imaging. Mice were anesthetized with isoflurane and bioluminescence was measured with the IVIS In Vivo System (Caliper Life Sciences).

### Immunohistochemistry

Prior to euthanasia, mice were injected IP with Hypoxyprobe (Hypoxyprobe) according to the manufacturer's instructions. After excision, tumors were photographed, measured, and fixed in 4% formaldehyde for routine processing and embedding. Four micron thick sections were cut and stained with haematoxylin—eosin. For light microscopic immunohistochemistry, paraffin sections were cut at 4 μm. Slides were deparaffinized using xylene and absolute ethanol, rinsed in distilled water, exposed to H₂O₂ for 5 minutes or antigen unmasking. The antigen unmasking solution (citrate buffer, Thermo Scientific) was heated in a steamer to 105° C. for 10 minutes, then cooled to room temperature. The sections were rinsed with PBS IHC for 2 minutes (CELL Marque) and blocked with CAS-Block for 5 minutes (Invitrogen). Slides were rinsed with PBS IHC and reacted with primary antibodies targeting CD34 (Abcam), and FITC-MAb1 (Hypoxiporbe) followed by a rinse in PBS IHC and reaction with secondary antibodies: MACH-2 rabbit HRP-Polymer (Biocare Medical) and HRP linked to rabbit anti-FITC, accordingly. Sections were rinsed with PBS IHC and incubated with AEC (CELL Marque) for 10 minutes. Sections were rinsed in distilled water and PBS IHC and counterstained with Mayer's hematoxylin, and coverslipped with a permanent mounting medium (AquaSlip, American MasterTech).

Slides were photographed with a Nikon ECLIPSE Ti microscope. A grid of 19×14 square regions of interest (ROIs) measuring 405×405 micrometers each covering the entire scanned image. A mask of the secondary antibody pixels was created using ImagePro 9 (Media Cybernetics) and the number of pixels per ROI was counted. Pixel count from matching ROIs was correlated between endothelial cells and hypoxia over the entire section for each slide. ROIs with a hypoxia pixel count over the background level were labeled “hypoxic” and the rest were labeled “normoxic”.

### Statistical Analyses

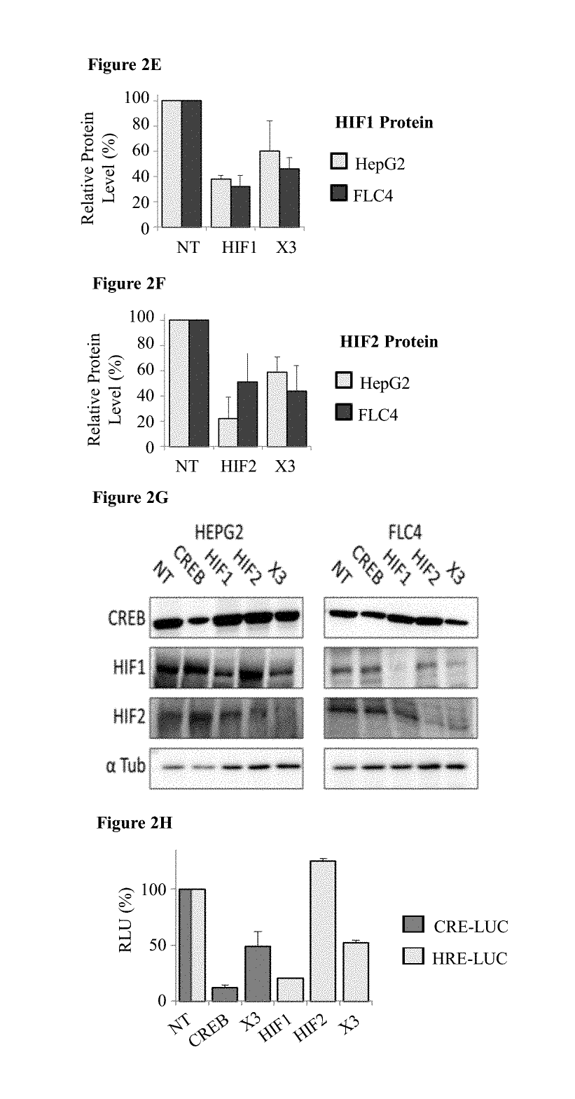

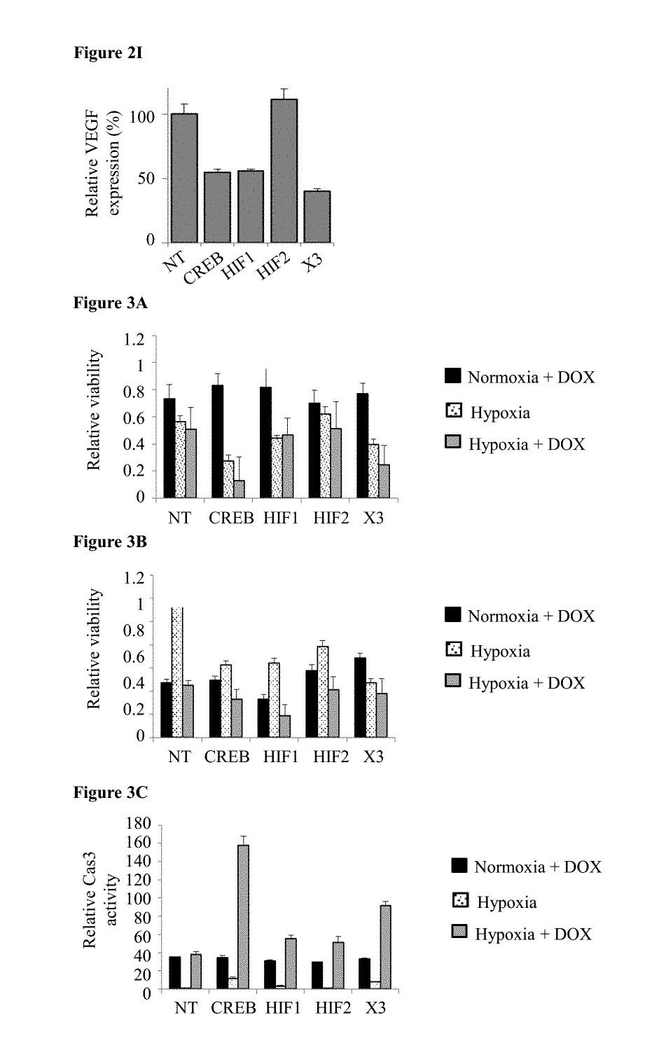

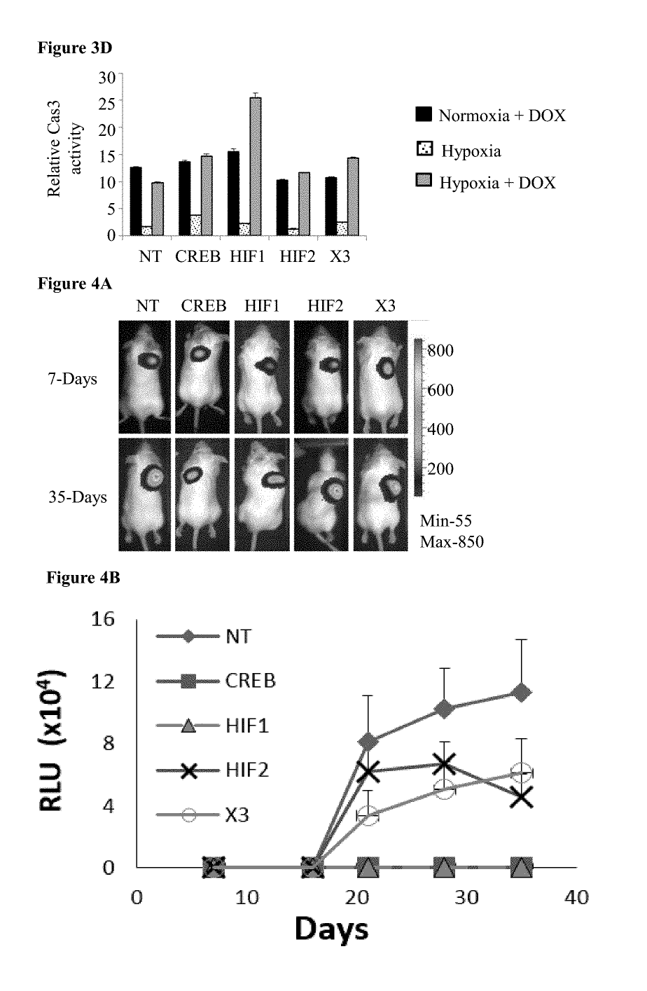

Statistical analysis was performed with JMP 9.0 (SAS). Analysis of variance (ANOVA) was used to compare mRNA levels, luciferase activity in relative light units, and the relative VEGF ELISA expression levels. Multivariate ANOVA for repeated measures was used to compare the cell viability, caspase-3 activity (FIG. 3), and the growth rates of the tumors within the mice (FIG. 4). ANOVA was used to compare viability and caspase-3 activity levels at the last time point (FIGS. 3, 7, and 8). A pairwise correlation between CD34 and HypoxyProbe pixel counts was performed and the correlation coefficient was compared between hypoxic and normoxic ROIs (FIG. 5).

### Example 1

### Construction and Functional Analyses of MuLV Replication Competent Viruses (RCR) Expressing shRNA Targeting Hypoxia Responsive Transcription Factors

Two properties of tumors—propagation of tumor cells and generation of hypoxic regions within the growing tumors—were exploited to construct a system that will preferentially lead to the death of tumor cells and thus hinder tumor growth.

Two major features that characterize tumors are tumor cell replication and on-going development of hypoxic regions. With these two characteristics in mind, vectors were constructed that infect only dividing cells and harm only hypoxic cells. To achieve this goal, MuLV based RCRs expressing shRNAs targeting the major regulators of the cellular responses to hypoxia were constructed. More specifically, the GFP coding region in the plasmid vector pACE-GFP was replaced with an H1 promoter and sequences of shRNAs targeting either CREB, HIF-1 or HIF-2 (pACE-CREB, pACE-HIF1 and pACE-HIF2, respectively) or transcribing a non-target sequence (pACE-NT) as a control. Because MuLV infected cells cannot be re-infected by MuLV, in order to knockdown all three regulators in each cell, a sequence harboring a polycistronic RNA molecule coding for all three shRNAs (pACE-X3) (FIG. 1) was cloned.

To determine if infection with the recombinant RCRs will generate a knockdown infection, viral particles were made by transfection of HEK293T cells with the recombinant plasmid vectors (see above) and the virus particles were collected with the growth media 48 h post-transfection. Based on monitoring GFP producing cells following infection by vACE-GFP (results not shown), both HepG2 and FLC4 cells were infected for about two weeks to reach a fully infected culture. The efficiency of knockdown of HIF-1, HIF-2 or CREB, was determined by RT-qPCR and Western blot analyses. The assays were carried out following incubation of the cells for 24 h in hypoxic conditions to determine the knockdown of HIF-1 and HIF-2. Since infection with non-target shRNA (vACE-NT) did not change significantly the expression of any of the three tested genes relative to non-infected cells, knockdown efficiencies by the other viruses was compared to cells infected with vACE-NT and to a control gene, GAPDH.

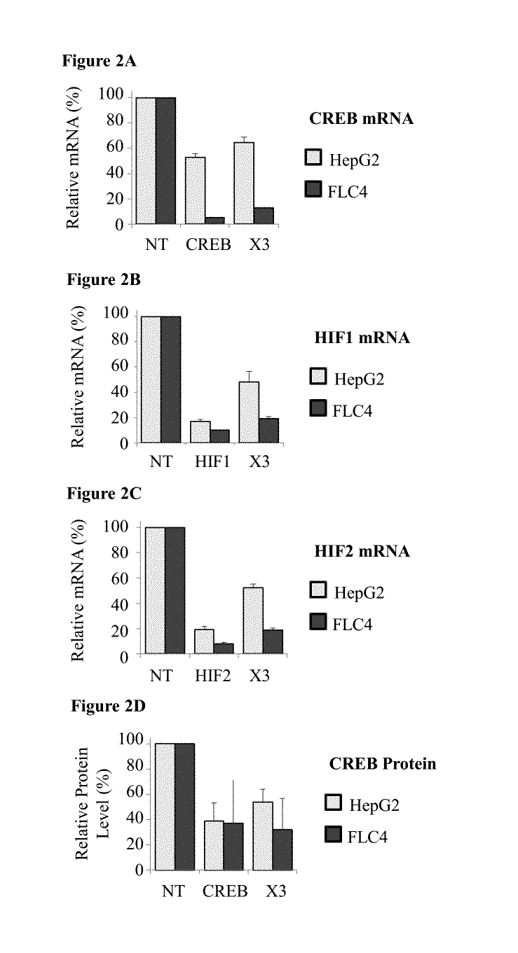

The levels of CREB mRNA in HepG2 and FLC4 cells infected with vACE-CREB was reduced by 65% and 95% (FIG. 2A) and the CREB protein by 61% and 63%, respectively (FIG. 2D, 2G). The knockdown of HIF-1 and HIF-2 in cells infected with either vACE-HIF1 or vACE-HIF2 was very efficient in HepG2 cells: 81-83% reduction of the mRNA (FIG. 2B-C) and 62% and 78% of the HIF-1 and HIF-2 proteins (FIG. 2E-G). A similar reduction was found in FLC4 cells: 90% and 92% of mRNA (FIG. 2B-C) and 68% and 49% of HIF-1 and HIF-2 proteins (FIG. 2E-G), respectively Although less efficient than in cells infected with RCR targeting each gene individually, infection with the RCR expressing the multi-shRNA, vACE-X3 resulted in a reduction of CREB mRNA (FIG. 2A) by 47% and 95% and the CREB protein (FIG. 2D, 2G) by 66% and 68%, for HepG2 and FLC4, respectively. The reduction of HIF-1 and of HIF-2 mRNAs (FIG. 2B-C) and proteins (FIG. 2E-G) in vACE-X3 infected HepG2 cells was 52% and 48% in the mRNA of both genes and 40% and 41% in the protein level, respectively. In FLC4 vACE-X3 infected cells, the reduction of HIF-1 mRNA (FIG. 2B) and protein level (FIG. 2E, 2G) 81% and 54%, respectively. The reduction of HIF-2 mRNA (FIG. 2C) and protein level (FIG. 2F-G) was 81% and 56%, respectively.

### Example 2

### The Effect of the Knockdown of HIF-1, HIF-2 and CREB on the Expression of CRE and HRE Mediated Gene Expression

To monitor the effect of the virus-mediated knockdown of CREB, HIF-1 and HIF-2 on activation of downstream genes in the stably infected HepG2 cells with any of the RCR viruses (vACE-CREB, vACE-HIF1, vACE-HIF2 or vACE-X3) cells were transfected with either plasmid pCREluc, CRE-mediated luciferase gene expression or pHREluc in which luc gene expression is activated by either HIF-1 or HIF-2. Luciferase activity was determined in normoxia and hypoxia 48 h post transfection. As expected, reduction of 61% in CREB or 62% in HIF-1 proteins resulted in reduction of 88% and 80% in CRE or HRE-mediated luc activity, respectively (FIG. 2H). In cells infected with vACE-X3, CRE or HIRE-mediated Luc activity was reduced by more than 50% (FIG. 2H). This result correlates with the less efficient knockdown of CREB and HIF-1 by vACE-X3 relative to the viruses expressing either one of the shRNA individually. Knocking down HIF-2 did not reduce the expression of HIRE-mediated luc relative to cells infected with vACE-NT (FIG. 2H). Because HIF-1 and HIF-2 recognize the same activation domain, EIRE, the HRE-mediated luc expression in cells infected with vACE-HIF-2 might have been activated by the more efficient HIF-1.

### Example 3

### The Role of HIF-1, HIF-2 and CREB in the Secretion of Endogenous VEGF in Hypoxia

In response to hypoxia, solid tumors stimulate tumor angiogenesis through HIF-induced expression of proangiogenic factors. One of the HIF-1 and CREB-activated proangiogenic growth factors is the vascular endothelial growth factor (VEGF).

To monitor the effect of CREB, HIF-1 and 2 on VEGF in stably infected HepG2 cells with one of the four RCR viruses (vACE-CREB, vACE-HIF1, vACE-HIF2 or vACE-X3) the cells were cultured in normoxic and hypoxic conditions for 24 hours.

Targeting either CREB or HIF-1 (vACE-CREB, vACE-HIF-1) diminished VEGF expression in HepG2 cells by 45% each (FIG. 2I) at hypoxia vs. normoxia as measured by ELISA. However, targeting HIF-2 had only a minor impact on VEGF expression in these cells. Targeting all three genes with the vACE-X3 showed a combined effect reducing the expression of VEGF by 58% (FIG. 2I). This result is consistent with the finding that both CREB and HIF-1 regulate VEGF expression in hypoxia.

### Example 4

### The Role of CREB, HIF-1, and HIF-2 in Protection of HepG2 Cells from Hypoxia Induced Apoptosis

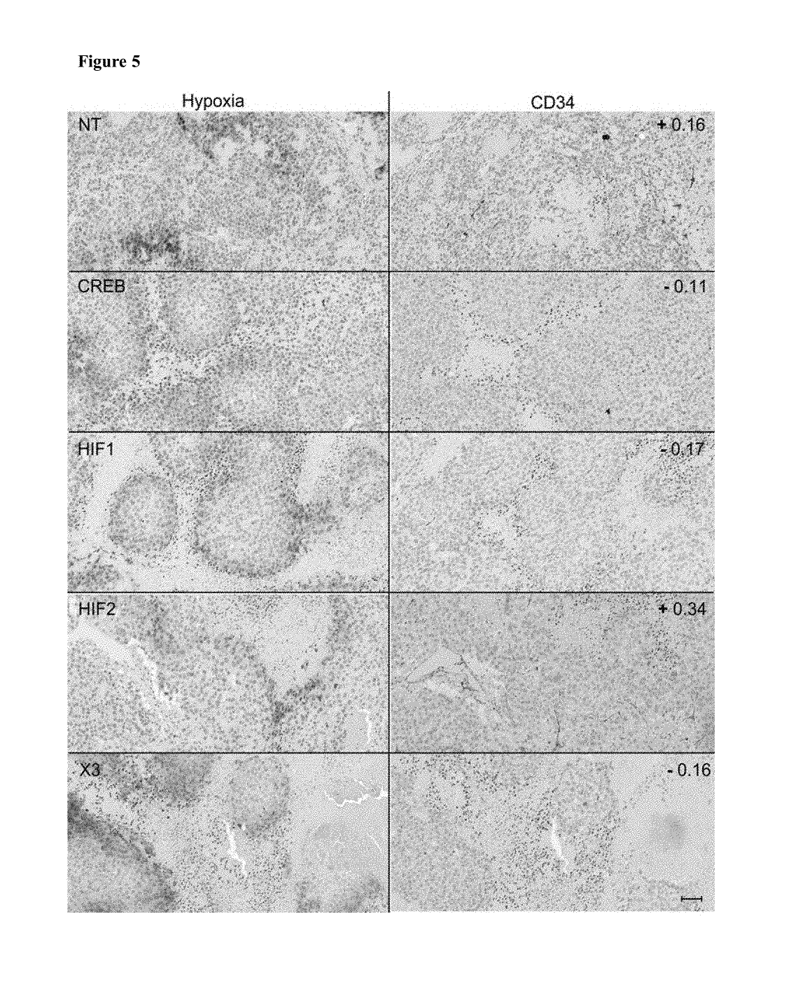

The contribution of each of the three transcription factors on the survival of HepG2 and FLC4 cells in hypoxia was further assessed. Cells stably infected with MuLV expressing shRNAs targeting CREB, HIF-1, HIF-2 or with the virus expressing the polycistronic shRNA cassette (X3) were incubated for 72 h under either normoxic or hypoxic conditions. At different time points during cells growth, cell viability, relative to time zero and caspase-3 activation relative to living cells at each time point and to time zero were determined (FIG. 7A-C, 8A-C). After 72 h of hypoxia, only about 27% of HepG2 (FIG. 3A, 7A) and 62% FLC4 cells (FIG. 3B, 8A) infected with vACE-CREB survived, while in control cells (cells infected with vACE-NT) 56% of HepG2 cells survived and no death was observed in FLC4 cells (FIG. 3A-B). Knockdown of HIF-1 had a smaller effect than knockdown of CREB on the survival of HepG2 cells in hypoxia (44% vs. 27% survived). Knockdown of either CREB or HIF1 in infected FLC4 had a similar effect on their survival (62%) after 72 h of hypoxia (FIG. 3B). Exposure of HepG2 or FLC4 cells infected with vACE-HIF2 to 72 h of hypoxia had no significant effect on survival of these cells or on the activation of caspase-3 relative to cells infected with vACE-NT (FIG. 3C-D, 7A, 8A). The increase in cell mortality of cells (2 times greater) with diminished CREB levels relative to cells infected with vACE-NT correlates with the increase (11 times greater) in activated caspase-3 in HepG2 cells and (3.8 times greater) in FLC4, relative to time zero. Knockdown of HIF-1 had a lesser but still significant effect on the activation of caspase-3 in the cells (FIG. 3C-D, 7A, 8A). In HepG2 and FLC4 cells infected with vACE-X3 the survival was similar (40 and 46%) and the activation of caspase-3 were somewhat less than in cells infected with vACE-CREB in both cell lines. The importance of CREB for the survival of these cells in hypoxia was noticeable already in early hypoxia (up to 24 h) in HepG2 cells (FIG. 7A).

The results presented here indicate that CREB, more than HIF-1 and HIF-2, plays a pivotal role in the survival of both HepG2 and FLC4 in hypoxic conditions in vitro.

### Example 5

### The Effect of Combined Treatment by RCR Mediated Knockdown of Hypoxia Responding Control Elements and Drug Treatment on Cell Survival

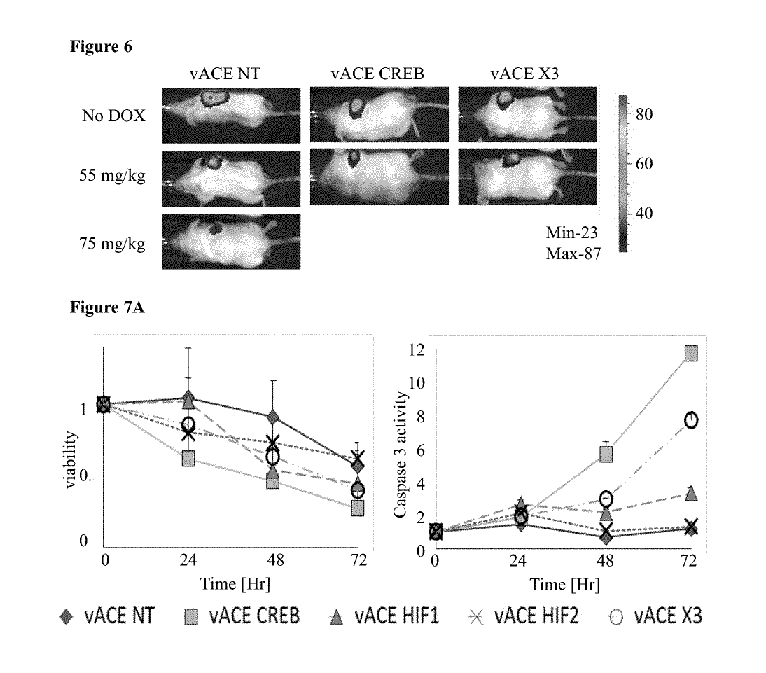

Doxorubicin (DOX)-induced tumor cell death has been linked with both CREB and HIF-1 pathways. Thus, it was hypothesized that combining the knockdown of the hypoxia responding factors with DOX treatment would have a synergistic effect and may reduce the effective clinical dose of DOX and thus diminish the side effects of the drug. To test this hypothesis survival of RCR infected HCC cells treated with varying concentrations of DOX was measured and the minimal lethal dose of DOX on HepG2 and FLC4 tumor cells was determined. The cells were treated with increasing concentrations of DOX and cultivated in normoxic or hypoxic conditions. Knowing the minimal lethal dose, HepG2 (FIG. 7B-C) and FLC4 cells (FIG. 8B-C) expressing the various shRNAs, were treated with 1 μm DOX, the minimal concentration that diminished the growth of the treated cells in normoxia. Cell viability and caspase-3 activation following treatment with DOX were similar in cells infected with the various RCRs in normoxia (FIG. 3 black columns and FIG. 7B, 8B).

In normoxic conditions, 72 h after administering DOX, cell viability decreased by 27% and by 53% in HepG2 (FIG. 3A) and FLC4 cells (FIG. 3B) infected with vACE-NT, respectively. At the same time caspase-3 activation in HepG2 (FIG. 3C) and in FLC4 cells (FIG. 3D) increased by 35 and 12-fold, respectively. At normoxia knockdown of CREB, HIF1 and HIF2 did not contribute to cell death or caspase-3 activation induced by DOX.

As described above after 72 hr of hypoxia, knockdown of CREB reduced the viability the two HCC cell lines such that 73% of HepG2 and of 38% of FLC4 died, relative to time zero. Combining hypoxia with DOX treatment on these infected cells resulted in cell death for 87% of HepG2 and 68% of FLC4 infected cells (FIG. 3A-B solid grey bars, and FIG. 7C, 8C). Increased caspase-3 activity correlated with the mortality of the treated cells (FIG. 3C-D). FLC4 cells showed a milder response than HepG2 to the combined treatment of CREB knockdown and DOX in hypoxic conditions. The effect of DOX treatment of HepG2 cells and FLC4 infected with vACE-CREB in hypoxia was, respectively, 5 and 1.5 times higher than the effect of treatment on vACE-NT treated cells.

Knockdown of HIF1 in HepG2 cells had no additional effect in combination with DOX in normoxia. In contrast, knockdown of HIF-1 in FLC4 in combination with DOX showed a 67% reduced viability in normoxia with a matching increase in activated caspase-3 levels. In hypoxia FLC4 cells where more sensitivity than HepG2 to the combined treatment of HIF 1 knockdown and DOX. In hypoxia, only 18% of the vACE-HIF1 infected FLC4 cells survived the combined treatment, while 46% of the HepG2 infected cells survived the treatment, similar to HepG2 cells infected with vACE-NT (50%). Knockdown of HIF2 did not affect the outcome of treatment of these two cell lines with DOX, neither at normoxia or hypoxia.

HepG2 cells infected with vACE-X3, expressing all 3 shRNAs, resulted in a similar, although milder, effect on survival and caspase-3 activity in hypoxia as was seen with knockdown of only CREB (vACE-CREB). Only a minor effect on mortality of DOX treated FLC4 infected with vACE-X3 was noticed (FIG. 3B). These results may be due to a lower activity of the shRNAs in vACE-X3 relative to viruses expressing each shRNA individually (FIG. 2A-C).

Put together, HCC cells infected with the RCR vectors expressing the various shRNAs increase the sensitivity of cells to DOX treatment.

### Example 6

### Treating Uveal Melanoma (UM)

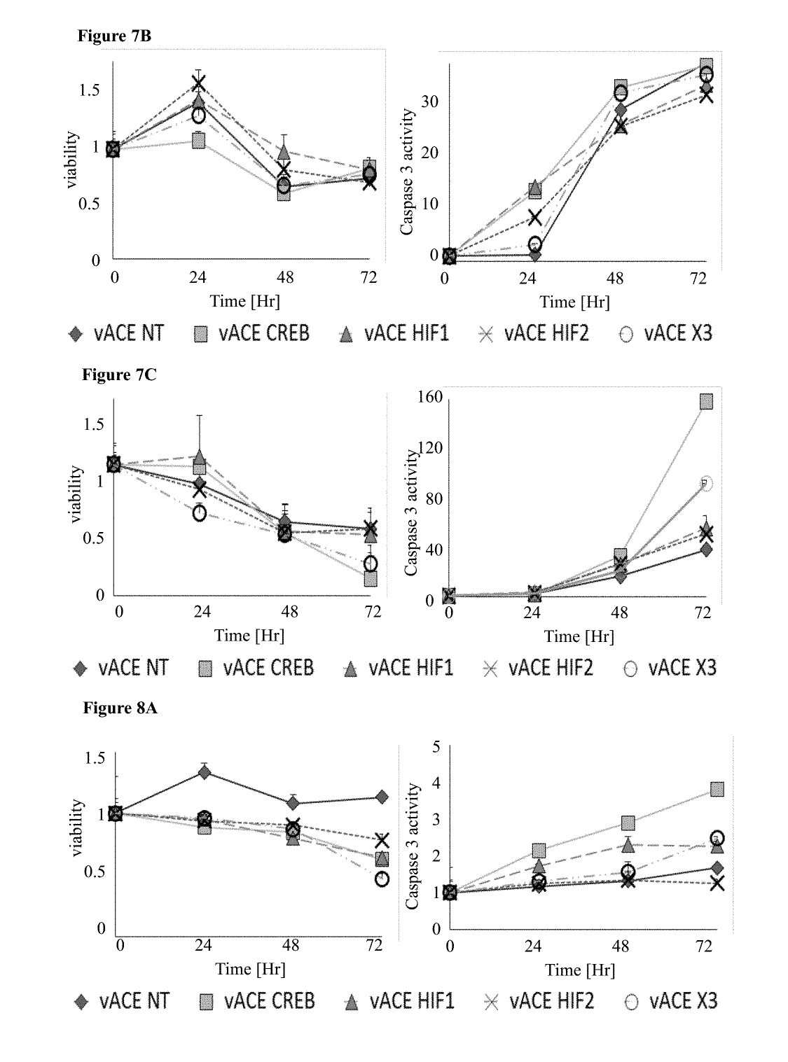

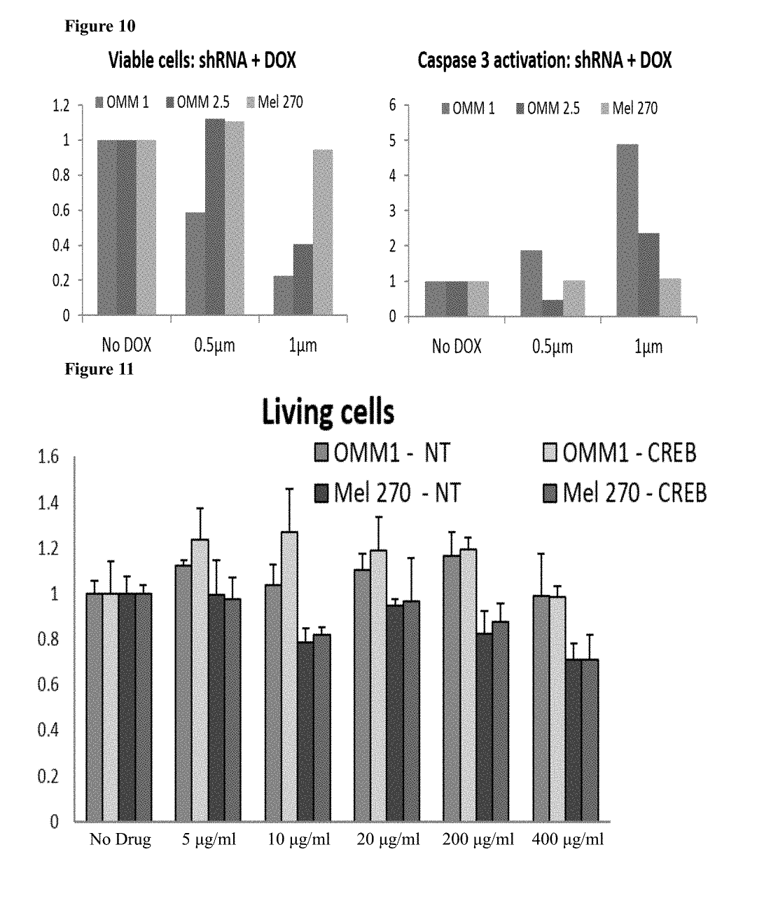

The results presented here indicate that CREB plays a pivotal role in the survival of HepG2 cells in hypoxic conditions in vitro. The further experiments were conducted on several UM cell lines (Mel270, OMM2.3, OMM2.5, OMM1, and 92.1). The aggressive primary uveal melanoma cell line Mel270, and its metastases OMM2.3 and OMM 2.5, along with 92.1, and OMM1 were stably infected with vACE-CREB which knocked-down the expression of CREB as determined by real-time RT-PCR, and the activity of downstream genes. The proliferation of infected/transfected UM cells was dramatically inhibited by de-novo knock-down of CREB. Specifically, vACE-CREB infected uveal melanoma cells treated with DOX showed a synergistic effect on cellular death induction and induction of caspase 3. (FIG. 9, 10). Likewise, vACE-CREB infected uveal melanoma cells treated with DTIC show a synergistic effect and induction of caspase 3 (FIG. 11, 12).

In conclusion, Doxorubicin proved to induce cell death in uveal melanoma cells. Cell lines derived from metastases are more sensitive than lines derived from primary tumors. Infectious knock-down of CREB via RCRs increased the sensitivity of the tumor cells to DOX (possibly more so in hypoxic conditions) and to DTIC. This treatment is complementary to targeted (blood-born) delivery methods of DOX. Increasing sensitivity to DOX substantially helps reducing the administered doses and reduce the risk for cardiotoxicity.

### Example 7

### The Effect of Knockdown of CREB, HIF-1, and HIF-2 on Tumor Growth

SCID mice were inoculated subcutaneously with 4×10⁶ HepG2 cells stably infected with either vACE-NT, vACE-CREB, vACE-HIF1, vACE-HIF2 or vACE-X3. The rate of growth of the tumors was monitored by a IVIS in-vivo camera system. The results presented in FIG. 4A clearly demonstrate that knockdown of either CREB or HIF-1 abrogates the tumor growth. In agreement with the in-vitro results (FIG. 3A), tumor growth of cells infected with vACE-X3 was affected less than tumors infected with either vACE-CREB or vACE-HIF1 (FIG. 4A-B). Although no effect on tumor cell survival was noticed in vitro following knockdown of HIF-2, in vivo knockdown of HIF-2 did moderately affect the growth rate of the HepG2 tumors in mice (FIG. 4B).

### Example 8

### Effect of Knockdown of CREB, HIF-1, and HIF-2 on VEGF and Blood Vessels Distribution in HCC Growing Tumors

For histopathologic analyzes tumors were excised from the mice after 35 days. The hypoxic regions were detected by Hypoxyprobe technology, and VEGF and blood vessels were detected by antibodies targeting VEGF or CD34 respectively. In agreement with the in vitro result presented FIG. 2I, the expression of VEGF in tumors harboring vACE-CREB, vACE-HIF1 or vACE-X3 was reduced dramatically (FIG. 4C). This result, in correlation with the finding that both CREB and HIF-1 are essential for activation of VEGF expression, demonstrates that infection of HCC tumor cells with vACE-CREB, vACE-HIF1 and vACE-X3 may serve to abolish hypoxia-mediated neovascularization in growing tumors. Similar to the in vitro experiments, knockdown of HIF-2 had no effect on VEGF expression in the growing HCC xenografts.

The reduction in VEGF expression by targeting CREB and/or HIF-1 is expected to diminish blood vessels growth toward the hypoxic regions. Indeed, CD34 stained blood vessels (FIG. 5, right panels, marked red) directed towards the hypoxic regions (FIG. 5, left panels, marked red) in tumors infected with vACE-NT or with vACE-HIF2. However, only scant vessels were noticed in tumors infected with vACE-CREB, vACE-HIF1, or vACE-X3. This was highlighted by correlating the amount of blood vessel staining to the level of hypoxia in these tumors (marked on the upper right corner of the right column of FIG. 5) with a positive correlation between CD34 and hypoxia in tumors infected with vACE-NT or vACE-HIF2 (0.16 and 0.34, respectively), and a negative correlation in tumors infected with vACE-CREB, vACE-HIF1, or vACE-X3 (−0.11, −0.17, and −0.16, respectively).

### Example 9

### Effect of Combined Treatment on Tumor Growth

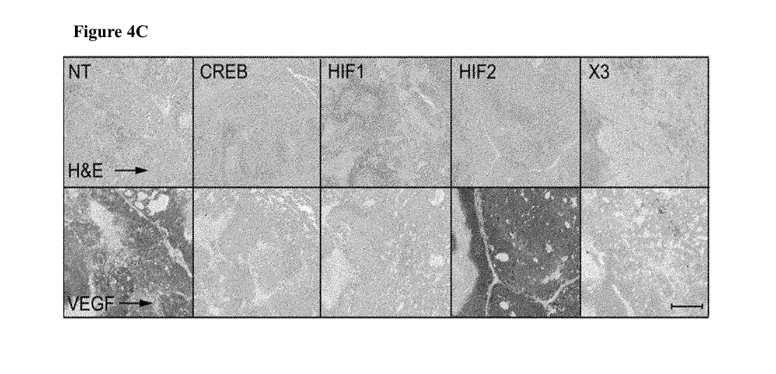

SCID mice were inoculated subcutaneously with HepG2 cells stably infected with either vACE-NT, vACE-CREB, or vACE-X3. Tumors were allowed to grow for two weeks, and then mice were injected intraperitoneally with DOX (20-75 mg/kg) twice a week (data not shown). A concentration of 75 mg/kg was sufficient to almost completely eradicate the tumors even in vACE-NT infected tumors (FIG. 6, bottom row). To assess combined effects a sub-lethal dose (55 mg/kg) was used. At six weeks, similar to the in vitro findings above, tumors infected with either vACE-CREB or vACE-X3 were slower to grow (FIG. 6, top row). The addition of DOX reduced the growth rate of all tumors. Tumor growth measured by light emission was compared between day 42 and day 14 post inoculation. The growth rate of DOX-treated tumors infected with vACE-NT was reduced by 45%. The combined treatment of infection with either vACE-CREB or vACE-X3 resulted in about 92% and 94% reduction in tumor growth respectively (FIG. 6, middle row).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of inducing cell death in a proliferating cell, the method comprising:
a. contacting said cell with a replication competent retrovirus (RCR) comprising one or more antisense molecules that target at least one hypoxia-inducible gene selected from the group consisting of: HIF-1 and CREB, and
b. exposing said cell to an anti-cancer therapy,
thereby inducing cell death in a proliferating cell.

2. The method of claim 1, wherein said RCR comprises an antisense molecule that targets at least two hypoxia-inducible genes selected from the group consisting of: HIF-1, HIF-2 and CREB.

3. The method of claim 1, wherein said RCR comprises an antisense molecule that targets HIF-1, HIF-2 and CREB.

4. The method of claim 1, wherein said RCR comprises a nucleic acid sequence selected from the group consisting of: GAGAGAGGTCCGTCTAATG (SEQ ID NO: 14), CTAACTGGACACAGTGTGTTT (SEQ ID NO: 15), and CTAACTGGACACAGTGTGTTTAATATATGAAAACACACTGTGTCCAGTTAGTAAGT CGACTCGCTTATTAAAGTATTCTGATCCGATTATAAAGGATCAGAATACTTTAATA AGAATGGCGCGTCTTCGAGAGAGGTCCGTCTAATG (SEQ ID NO: 16).

5. The method of claim 1, wherein said retrovirus is a Murine Leukaemia virus (MuLV).

6. The method of claim 1, wherein said anti-cancer therapy comprises a therapy selected from the group consisting of: radiation therapy, chemotherapy, immunotherapy, and any combination thereof.

7. The method of claim 6, wherein said anti-cancer therapy is a chemotherapy comprising administering a chemotherapeutic agent selected from the group consisting of: Doxorubicin and Dacarbazine.

8. The method of claim 1, wherein said proliferating cell is a cancerous cell.

9. A method of treating, or ameliorating cancer in a subject in need thereof, the method comprising:
a. administering to said subject a replication competent retrovirus (RCR) comprising one or more antisense molecules that target at least one hypoxia-inducible gene selected from the group consisting of: HIF-1 and CREB, and
b. administering to said subject an anti-cancer therapy,
thereby treating or ameliorating cancer in a subject in need thereof.

10. The method of claim 9, wherein said RCR comprises an antisense molecule that targets at least two hypoxia-inducible genes selected from the group consisting of: HIF-1, HIF-2 and CREB.

11. The method of claim 9, wherein said RCR comprises a nucleic acid sequence selected from the group consisting of: GAGAGAGGTCCGTCTAATG (SEQ ID NO: 14), CTAACTGGACACAGTGTGTTT (SEQ ID NO: 15), and CTAACTGGACACAGTGTGTTTAATATATGAAAACACACTGTGTCCAGTTAGTAAGT CGACTCGCTTATTAAAGTATTCTGATCCGATTATAAAGGATCAGAATACTTTAATA AGAATGGCGCGTCTTCGAGAGAGGTCCGTCTAATG (SEQ ID NO: 16).

12. The method of claim 9, wherein said retrovirus is a Murine Leukaemia virus (MuLV).

13. The method of claim 9, wherein said anti-cancer therapy comprises a therapy selected from the group consisting of: radiation therapy, chemotherapy, immunotherapy, and any combination thereof.

14. The method of claim 13, wherein said chemotherapy comprises a chemotherapeutic agent selected from the group consisting of: Doxorubicin and Dacarbazine.

15. The method of claim 9, wherein said cancer is a solid tumour.

16. The method of claim 9, wherein said cancer is selected from the group consisting of: hepatoma, melanoma, liver cancer, epithelial cancer, carcinoma and hepatocellular carcinoma.

17. The method of claim 9, wherein said cancer is selected from uveal melanoma and hepatocellular carcinoma (HCC).

18. The method of claim 9, wherein administering said RCR is prior to, or together with, administering said anti-cancer therapy.

19. The method of claim 9, wherein administering said RCR potentiates at least one anti-cancer effect of said anti-cancer therapy.

20. A kit comprising at least one agents selected from the group consisting of:
a. a replication competent retrovirus (RCR) comprising at least one antisense molecule that targets a hypoxia-inducible gene selected from the group consisting of: HIF-1 and CREB, the RCR being adapted or identified for co-administration with an anti-cancer agent; and
b. an anti-cancer agent, adapted or identified for co-administration with an RCR.


SEQUENCE LISTING (24 sequences)

SEQ ID NO: 1 - DNA, 52 bp, Artificial
  Synthetic
gagagaggtccgtctaatgttcaagagacattagacggacctctctcttttt

SEQ ID NO: 2 - DNA, 57 bp, Artificial
  Synthetic
ctaactggacacagtgtgtttaatatatgaaaacacactgtgtccagttagtttttt

SEQ ID NO: 3 - DNA, 54 bp, Artificial
  Synthetic
attaaagtattctgatccgattataaaggatcagaatactttaataagtttttt

SEQ ID NO: 4 - DNA, 181 bp, Artificial
  Synthetic
ctaactggacacagtgtgtttaatatatgaaaacacactgtgtccagttagtaagtcgactcgcttattaaagtattctgatccgattataaaggatcagaatactttaataagaatggcgcgtcttcgagagaggtccgtctaatgcctgaaccacattagacggacctctctctttttt

SEQ ID NO: 5 - DNA, 55 bp, Artificial
  Synthetic
accaagatgaagagcaccaacctgaaccattggtgctcttcatcttggttttttt

SEQ ID NO: 6 - DNA, 24 bp, Artificial
  Synthetic
cccagcacttcctacacagcctgc

SEQ ID NO: 7 - DNA, 28 bp, Artificial
  Synthetic
cgagctgcttcctgttcttcattagacg

SEQ ID NO: 8 - DNA, 26 bp, Artificial
  Synthetic
gggattaactcagttgaactaactgg

SEQ ID NO: 9 - DNA, 27 bp, Artificial
  Synthetic
cctttttcacaaggccatttctgtgtg

SEQ ID NO: 10 - DNA, 23 bp, Artificial
  Synthetic
acaaggtgtcaggcatggcaagc

SEQ ID NO: 11 - DNA, 24 bp, Artificial
  Synthetic
cgttcacctcacagtcatatctgg

SEQ ID NO: 12 - DNA, 22 bp, Artificial
  Synthetic
ccatcttccaggagcgagatcc

SEQ ID NO: 13 - DNA, 22 bp, Artificial
  Synthetic
gcaaatgagccccagcttctcc

SEQ ID NO: 14 - DNA, 19 bp, Artificial
  Synthetic
gagagaggtccgtctaatg

SEQ ID NO: 15 - DNA, 21 bp, Artificial
  Synthetic
ctaactggacacagtgtgttt

SEQ ID NO: 16 - DNA, 147 bp, Artificial
  Synthetic
ctaactggacacagtgtgtttaatatatgaaaacacactgtgtccagttagtaagtcgactcgcttattaaagtattctgatccgattataaaggatcagaatactttaataagaatggcgcgtcttcgagagaggtccgtctaatg

SEQ ID NO: 17 - DNA, 21 bp, Artificial
  Synthetic
cttattaaagtattctgatcc

SEQ ID NO: 18 - DNA, 4059 bp, Human
gcgcgcgccggcctgggcaggcgagcgggcgcgctcccgccccctctcccctccccgcgcgcccgagcgcgcctccgcccttgcccgccccctgacgctgcctcagctcctcagtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggc

SEQ ID NO: 19 - DNA, 3932 bp, Human
gcgcgcgccggcctgggcaggcgagcgggcgcgctcccgccccctctcccctccccgcgcgcccgagcgcgcctccgcccttgcccgccccctgacgctgcctcagctcctcagtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggc

SEQ ID NO: 20 - DNA, 3956 bp, Human
atttgaaaacttggcaaccttggattggatggattcatatttcttagtatagaagttcttgatataactgaaaaattaagttaaacacttaataagtggtggttactcagcacttttagatgctgtttataatagatgaccttttctaactaatttacagttttttgaaagataactgagaggttgagggacggagattttcttcaagcaattttttttttcattttaaatgagctcccaatgtcggagtttggaaaacaaatttgtctttttaaaagaaggtctaggaaactcaaaacctgaagaattggaagaaatcagaatagaaaatggtaggataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggc

SEQ ID NO: 21 - DNA, 5166 bp, Human
gctttacactcgcgagcggaccgccacacgggtccggtgcccgctgcgcttccgccccagcgctcctgaggcggccgtacaatcctcggcagtgtcctgagactgtatggtcagctcagcccggcctccgactccttccgactcccagcattcgagccacttttttttttctttgaaaactcagaaaagtgactccttttccagggaaaaaggaacttgggttcccttctctccgtcctcttttcgggtctgacagcctccacccactccttccccggaccccgcctccgcgcgcaggttcctcccagtcacctttctccacccccgcccccgcacctagcccgccgcgcgccaccttccacctgactgcgcggggcgctcgggacctgcgcgcacctcggaccttcaccacccgcccgggccgcggggagcggacgagggccacagccccccacccgccagggagcccaggtgctcggcgtctgaacgtctcaaagggccacagcgacaatgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctgagccaggccttctacctgggcagcacctctgccgacgccgtcccaccagcttcactctctccgtctgtttttgcaactaggtatttctaacgccagcacactatttacaagatggacttacctggcagacttgcccaggtcaccaagcagtggcctttttctgagatgctcactttattatccctatttttaaagtacacaattgttttacctgttctgaaatgttcttaaattttgtaggatttttttcctccccaccttcaatgacttctaatttatattatccataggtttctctccctccttctccttctcacacacaactgtccatactaacaagtttggtgcatgtctgttcttctgtagggagaagctttagcttcattttactaaaaagattcctcgttattgttgttgccaaagagaaacaaaaatgattttgctttccaagcttggtttgtggcgtctccctcgcagagcccttctcgtttcttttttaaactaatcaccatattgtaaatttcagggtttttttttttttgtttaagctgactctttgctctaattttggaaaaaaagaaatgtgaagggtcaactccaacgtatgtggttatctgtgaaagttgcacagcgtggcttttcctaaactggtgtttttcccccgcatttggtggattttttattattattcaaaaacataactgagttttttaaaagaggagaaaatttatatctgggttaagtgtttatcatatatatgggtactttgtaatatctaaaaacttagaaacggaaatggaatcctgctcacaaaatcactttaagatcttttcgaagctgttaatttttcttagtgttgtggacactgcagacttgtccagtgctcccacggcctgtacggacactgtggaaggcctccctctgtcggctttttgccatctgtgatatgccataggtgtgacaatccgagcagtggagtcattcagcgggagcactgcgcgctatcccctcacattctctatgtactatgtatgtatgtattattattattgctgccaagagggtctgatggcacgttgtggggtcggggggtggggcggggaagtgctctaacttttcttaaggttttgttgctagcccttcaagtgcactgagctatgtgactcggatggtctttcacacggcacatttggacatttccagaactaccatgagatggtttagacgggaattcatgcaaatgaggggtcaaaaatggtatagtgaccccgtccacgtcctccaagctcacgaccttggagccccgtggagctggactgaggaggaggctgcacagcgggagagcagctggtccagaccagccctgcagcccccactcagccggcagccagatggccccgcaaggcctccagggatggcccctagccacaggccctggctgaggtctctgggtcggtcagtgacatgtaggtaggaagcactgaaaatagtgttcccagagcactttgcaactccctgggtaagagggacgacacctctggtttttcaataccaattacatggaacttttctgtaatgggtacaatgaagaagtttctaaaaacacacacaaagcacattgggccaactatttagtaagcccggatagacttattgccaaaaacaaaaaatagctttcaaaagaaatttaagttctatgagaaattccttagtcatggtgttgcgtaaatcatattttagctgcacggcattaccccacacagggtggcagaacttgaagggttactgacgtgtaaatgctggtatttgatttcctgtgtgtgttgccctggcattaagggcattttacccttgcagttttactaaaacactgaaaaatattccaagcttcatattaaccctacctgtcaacgtaacgatttcatgaacgttattatattgtcgaattcctactgacaacattataactgtatgggagcttaactttataaggaaatgtattttgacactggtatcttattaaagtattctgatcctaccactg

SEQ ID NO: 22 - DNA, 10208 bp, Human
tgtttccgtgcgcggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaataaaaaaaatgtctcatttgggaaggaaattccttttaaaaaagagttgagacacttagaaaactaatgttttatatttagtcaagagttatttaagaaagtcaagcttgtttaacaacaaaatatgaagatttaagtgttaattgctggatccattttaaaataagattttaattaacatttgtaaatggtatattttcgtttgtaacaaaccattgtcttttttcaaggatgaacagagtttatgaaggagcatcattctaagaattaagtgatgtagtctttatgtttggacagttcaccagattctcaagaaggctttcaaacaactataaagtttgatgtttgtcctgctgagctaatggggaaagttatagcataaaaattgtgtaaccgcatagatatgtcatttttaaaaactggtttaacagaaatcaagcaaagtcacaaatatgttcacaagttggaattatttattgagtcaaaatgtcgaatcgaacattttgaatgaagtaagtgttataaatgaaaaattgcctgatgtttagcagtttgtattctctaaagctttttttcaaaagttcaggctttctacttactgggaagttggtggtcctcttagtccctgataaatcaaggcaatcacattcatgtgagctggatgaatttataagttataaagaccttatccttcataccttgaggatgattgcactggttttgaagtcagttgcttaatgatgaggtgagaaatgtatcctgttgctaaatctgtcttagacccttggtgaaacttgaagatttcagtttataaagataaaatcaagcatcttttgtgcagttttctttttttaatgcaagaatggtggggaggtttgtttgtaagcatgaaactttgagaatctttattaagaaaatgacataatttttaaaaaccttgtagccaagaacatatgtggccacattaccagtaataaatgtttttctctttatattggccaaaagggaataaaaatgtcatcataggaatttgtacatatgctactgatttgcctagaaaatagcaagtttgatattgctcactttgcaaatatagggccatgtggcacttttatctataggacagattaataaaaatgaagtggggaggggtttatttttgatatattactcttatgagttttcaagctttgataatgtttaactgaaaagtggcttagaaagggctagatccaatgtgttcattattaaataattgctatcagatacaattttaagttcattctttttcaaactcaagtaccatattggcaaccataatattgtcataggtgctctcttcatttagatattcttggggggggtggcatttgtataatatatgtgtacatatatatatatatatatatatatacatacagtatataatctaaagctctgagagctcttaagtcaggaatgctgagtattatagtatattgaggtcagatgaaattttacatttttgtgtgttctgttgcattccttctggtagtttctatgactgcattactccagcactcatgattgattttatcttctaattttcttccaagtattttattttttattagttttctttggcttgatacttttaaatatgttactagtcacttgaaagcctctcccccaaaagtatttggtttgtatgctttgtctgtggcagctataacagtggtaagaacattttgaagatagctttttaaaggaaccactgattttttcaaaaatcatcctgggggaggaattttggcatttcatttgagcagggattttgtcagaaaatgtgttttgatggtaggtcagcagcagtgctagtctctgaaagcacaataccagtcaggcagcctatcccatcagatgtcatctggctgaagtttatctctgtctctcaggataaatccctgtaggacaaatccctactatcatttctaccttttggggtgacatgtggaatcatacaaaggcttaggaagaaatacgtttgtttaaaccaggatgctttacttacttgaagtgacttcaatctagatttcttttaatatttaacaaatttttaattctatgatcagccacagtcagctattaccataaattggtctctgtttattttgaagatcacggctgcttcattttgcaggattaagtagggctaatgtatcttaaagttaagatcttgaattaaagtgagttttagaaatagtgttacataccttttcagttgttttcaagaggctttatttttgttgcctttgtagccctgaaagctgttggtatattttttccctcatggacccaatagaaaagttgtatatttatttggattatatttacattctgtcctttgtaaatgtttggtgtaacttgcacttttttaaatgacccagtttgggtattagcaacttaagaaattccctcatcaagtaattctcaactttttagtctttctcctctcttcaaatcatgtgactttttaaatggaagtttttcattgattaaaatattttagcacctaaaagctagccttaaaaacagctgtaaaagaaaaacatcaggaaattagatatgactagcccagttaattaaaagacgggctcaaaccttgttttattctttttcatcttggatgaagattgaagggaaaataactcaagtgcataatatttattttcaatttttaatgagactttatcctcatcacaacattaatactgtacatagtatgccaaaatatccattaatttgtctagaatagtacaagactttttaaagcaattgtcctcacagagaccacatgtaatatactgaaatatgttcatttttaatggctttgttaacatcaaagaaatgctgcctaaatttgatttcagatgaggaaggagaaagtaaagtgtgcatagtaaggctgtaggtgaagagttgtgagataaatagttcactcagttgtacaaagcacaactagaactttttgttgggaggcttacatacatcttgaatattcttaatgtaataatgttgactattaagttggctacacagtcactgtatgtactaggaactggtttccttgacattctagaatcaatggctaggagaggcattaatctttgaggggctgaacatatcatgaagctgagtcagtatggaaaattttcaaataaacagggtgctgaagttccatctgtctcatctgcttatgataagttcttattgattagtgaatgtagcttaagcctttgtatgtgtcctcagggggcagaccgactttaagagggaccagataacgtttgaatggagggattatatttcaggtgttttagcttgaaatttattttttaaaaaaagaaaaatttaaaaaatatataaataaaatagaacaaagccggtgatgcaagttgatattataaacaggcagttttagcacagaaagaaaatactgacctgtctgcattctggtacggtgggtgcaggtcccagctgggtatgacatgatacatttttaattattctcaccagcaagtaaaaggaaaatgaacaatcttttggaattgtctttgaaaaggatcaaagagtaggaaattcacatttgacctaacattacttgcctatagaagtatggcatttccaagcttttgtctgaggagcatctcagagaagtgagagtaaatctgagttagcttaaaaattggtagggaggaagaaaatctctgcaaataatgattttatgtttgttggccaagtgaaatgatctatcattgtgtttgggaggttttattttcttatgtttttaaaattggtaaatgctttatagatgtatttttatccaagtgccactccaatttgtgtatgtaataaaattatttatattaaaagtgggaaataattgtcaacattttttttgagtatagatttattaggggtggcaaagaagagtgctagttagcagttttccatgtaaagttgtccttgactgatttgtccacatgtcagttgtaactcccccactccctgcaaaaggaattatttctaacccagatgtatcacttgaaactttttagaagcaaaataatcagggaagttcctagaaaggtgtttggctttttggtttttgagggttggggtaaagaagacttcccccacaactgtcagcacaaaacagggtattgatttttaactctgatgtttctattggagttgaatactaaataaataactataatgagggaaatacatttctaataaaattccctacattctagaaacatccctgttttaatttttttatctaaatctttttgtgctttatgtgtaaagaaaaaaatgtactgagttacaatgcattttattaacactatgtacataatagctgctttgtgttcagaatagtagcagttgctttgtatattaaagtgatccttgtgaatttgtgaaatattgtcataaagtgctttttcttactgtaatctttgtggtatcaactgtcataatgctctttttacacaaacatttatgtgcagtcacataaacatgcttttaaaaactctgtaagtctcttttttggggatgggatctctatattttgttgggttttttttgctagtagtgtgaagccatgttttattggacttaaagttacaatatattacaagcttgtgttggaaggcagcaaaactaattcagacaacaacatgtcttcagttactggatccctaattttcaggacaaaacctgtttttcaataagattgaacagtgcctatttgtggatttggagatgttactgtcaagatgactaatggagacatacgaccagctgtgtctgatgtcataaaacacgtgttcactgaaaggacaataagactatataccttctcaggtccccttgcaattctaaaactctgtgatcatataaattggaaggaaaggggaggggatatggttaatctttgcttaagctgtaagaataaaaaagttatctcctatactattaacttctgaaataagttctgagacgagacatctgaaaataagcagctgcattatttgtatgtttcttcactgccaagatgtgttcaagcctgctatacctgccattgtattggaaggcttaatgaatttcatttattttctgcaacaacgattacagaatttattgcacaaaatgagacattttgagagtgatattaattacatgagggacaataggcatgaactaggattgttctaagcaaatcggaatcgggtcaccctgccacgttcaggtgcttggaccttcaggaaaagattgcccatcttgtcatttgaccaggcactgaagtgacaagaccatccttgagaagtcacatccaaagataaaattctgatccatttctagttttagtgtttcgccactgaagacttaacatatgtcttttacactcaggttgcaaaacacaggcccaagacaaacttaacttctcccccaaatcttccttccgctggtttttccatctcgtaagtggtgccactatccatctgttaaattgtttaggggaaacctagaaaagcactaccttaatcagtgttatccttcttcttaactgtgcgtcctaatttctccacatctttcttaagtgcagtgaccaaaccggatgagaattctaacacgggcctgacatcaaatggaaaggaaggataatgtccaggagttggaatgttatccttgtttttaattaagatgcaattcacataaattaactttttaagtgaacaattaagtggtagtacatccacaatggtgtacaaccaccacttctatctagctccaaaacattctcatcactccaaaagtaaagtcccgttactctccattttctcctcccaccgcccttgtccctggcaaccaccaatctgcttcctgtttctttggatttacatccgggtatttcatgtgagactcatacactgtgtattacttctttcgtctagctttaatgtgttgttgaggttgatccattgtaacatgttatcactacttcattcctttttatagctaagtatactttttatagtaagtatgccattgtagatatataccacaagtttatcgattcatccagttgagttgtttctactgtttggctaatgttcatagtgctgttatgaatgttcgtgtacaagtatttgagtccgtgttttcaattatttggggtatatgcctgggagtggagttgctgggtcatgttgaaatcgcacatttaactttttgaggaactgtcaaactttccctcagcagctgtaccgttttaccttccaccattgatgtatgagggttccaatttctccacaccttcaccaacacttattttgccattttaaaaattatagccatcctcatgggtgtggtctctcattgtggttttgatttgcatttccctgattactaatgatgtggagcatcttttgttgtctttggccatctgcgtatcttctttgaagaaatgtctgttgaggtcctttgttcattgaaattttgttgttgggttctgagttccttatatattctgggtactaggcccttataatattttcgcctataagtttttgctttataatgtcctcattgttttcaaacttactttatgtaatatgtacacttctaaaaaaaagaaacatggaaaagggcaaactgtaagaagttttttgtgttatgttttttgtgacagtctgtgcatatatacacaaatataatgtatgttctctcctcctccctctccctttttttacacaaaaggtaggtacaaacagtggtttataaactgctgccattgtacagatacagtttaaccagtcctcttctggggacatttggctgtttgaaattttttactgttacagatatacagaggttggtaactaggtctacacaagttgtatctccaggatactgagaagtaaaagttatttctgaattatggttttcttcatatttggatattgtttcctaatgattattaggtatctgctaagcaatttttattaacttatgttgattactatttttatgtcaaactttacagtctaggcatttttttctggaattaaaattagaagtggcacagac

SEQ ID NO: 23 - DNA, 10166 bp, Human
tgtttccgtgcgcggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaataaaaaaaatgtctcatttgggaaggaaattccttttaaaaaagagttgagacacttagaaaactaatgttttatatttagtcaagagttatttaagaaagtcaagcttgtttaacaacaaaatatgaagatttaagtgttaattgctggatccattttaaaataagattttaattaacatttgtaaatggtatattttcgtttgtaacaaaccattgtcttttttcaaggatgaacagagtttatgaaggagcatcattctaagaattaagtgatgtagtctttatgtttggacagttcaccagattctcaagaaggctttcaaacaactataaagtttgatgtttgtcctgctgagctaatggggaaagttatagcataaaaattgtgtaaccgcatagatatgtcatttttaaaaactggtttaacagaaatcaagcaaagtcacaaatatgttcacaagttggaattatttattgagtcaaaatgtcgaatcgaacattttgaatgaagtaagtgttataaatgaaaaattgcctgatgtttagcagtttgtattctctaaagctttttttcaaaagttcaggctttctacttactgggaagttggtggtcctcttagtccctgataaatcaaggcaatcacattcatgtgagctggatgaatttataagttataaagaccttatccttcataccttgaggatgattgcactggttttgaagtcagttgcttaatgatgaggtgagaaatgtatcctgttgctaaatctgtcttagacccttggtgaaacttgaagatttcagtttataaagataaaatcaagcatcttttgtgcagttttctttttttaatgcaagaatggtggggaggtttgtttgtaagcatgaaactttgagaatctttattaagaaaatgacataatttttaaaaaccttgtagccaagaacatatgtggccacattaccagtaataaatgtttttctctttatattggccaaaagggaataaaaatgtcatcataggaatttgtacatatgctactgatttgcctagaaaatagcaagtttgatattgctcactttgcaaatatagggccatgtggcacttttatctataggacagattaataaaaatgaagtggggaggggtttatttttgatatattactcttatgagttttcaagctttgataatgtttaactgaaaagtggcttagaaagggctagatccaatgtgttcattattaaataattgctatcagatacaattttaagttcattctttttcaaactcaagtaccatattggcaaccataatattgtcataggtgctctcttcatttagatattcttggggggggtggcatttgtataatatatgtgtacatatatatatatatatatatatatacatacagtatataatctaaagctctgagagctcttaagtcaggaatgctgagtattatagtatattgaggtcagatgaaattttacatttttgtgtgttctgttgcattccttctggtagtttctatgactgcattactccagcactcatgattgattttatcttctaattttcttccaagtattttattttttattagttttctttggcttgatacttttaaatatgttactagtcacttgaaagcctctcccccaaaagtatttggtttgtatgctttgtctgtggcagctataacagtggtaagaacattttgaagatagctttttaaaggaaccactgattttttcaaaaatcatcctgggggaggaattttggcatttcatttgagcagggattttgtcagaaaatgtgttttgatggtaggtcagcagcagtgctagtctctgaaagcacaataccagtcaggcagcctatcccatcagatgtcatctggctgaagtttatctctgtctctcaggataaatccctgtaggacaaatccctactatcatttctaccttttggggtgacatgtggaatcatacaaaggcttaggaagaaatacgtttgtttaaaccaggatgctttacttacttgaagtgacttcaatctagatttcttttaatatttaacaaatttttaattctatgatcagccacagtcagctattaccataaattggtctctgtttattttgaagatcacggctgcttcattttgcaggattaagtagggctaatgtatcttaaagttaagatcttgaattaaagtgagttttagaaatagtgttacataccttttcagttgttttcaagaggctttatttttgttgcctttgtagccctgaaagctgttggtatattttttccctcatggacccaatagaaaagttgtatatttatttggattatatttacattctgtcctttgtaaatgtttggtgtaacttgcacttttttaaatgacccagtttgggtattagcaacttaagaaattccctcatcaagtaattctcaactttttagtctttctcctctcttcaaatcatgtgactttttaaatggaagtttttcattgattaaaatattttagcacctaaaagctagccttaaaaacagctgtaaaagaaaaacatcaggaaattagatatgactagcccagttaattaaaagacgggctcaaaccttgttttattctttttcatcttggatgaagattgaagggaaaataactcaagtgcataatatttattttcaatttttaatgagactttatcctcatcacaacattaatactgtacatagtatgccaaaatatccattaatttgtctagaatagtacaagactttttaaagcaattgtcctcacagagaccacatgtaatatactgaaatatgttcatttttaatggctttgttaacatcaaagaaatgctgcctaaatttgatttcagatgaggaaggagaaagtaaagtgtgcatagtaaggctgtaggtgaagagttgtgagataaatagttcactcagttgtacaaagcacaactagaactttttgttgggaggcttacatacatcttgaatattcttaatgtaataatgttgactattaagttggctacacagtcactgtatgtactaggaactggtttccttgacattctagaatcaatggctaggagaggcattaatctttgaggggctgaacatatcatgaagctgagtcagtatggaaaattttcaaataaacagggtgctgaagttccatctgtctcatctgcttatgataagttcttattgattagtgaatgtagcttaagcctttgtatgtgtcctcagggggcagaccgactttaagagggaccagataacgtttgaatggagggattatatttcaggtgttttagcttgaaatttattttttaaaaaaagaaaaatttaaaaaatatataaataaaatagaacaaagccggtgatgcaagttgatattataaacaggcagttttagcacagaaagaaaatactgacctgtctgcattctggtacggtgggtgcaggtcccagctgggtatgacatgatacatttttaattattctcaccagcaagtaaaaggaaaatgaacaatcttttggaattgtctttgaaaaggatcaaagagtaggaaattcacatttgacctaacattacttgcctatagaagtatggcatttccaagcttttgtctgaggagcatctcagagaagtgagagtaaatctgagttagcttaaaaattggtagggaggaagaaaatctctgcaaataatgattttatgtttgttggccaagtgaaatgatctatcattgtgtttgggaggttttattttcttatgtttttaaaattggtaaatgctttatagatgtatttttatccaagtgccactccaatttgtgtatgtaataaaattatttatattaaaagtgggaaataattgtcaacattttttttgagtatagatttattaggggtggcaaagaagagtgctagttagcagttttccatgtaaagttgtccttgactgatttgtccacatgtcagttgtaactcccccactccctgcaaaaggaattatttctaacccagatgtatcacttgaaactttttagaagcaaaataatcagggaagttcctagaaaggtgtttggctttttggtttttgagggttggggtaaagaagacttcccccacaactgtcagcacaaaacagggtattgatttttaactctgatgtttctattggagttgaatactaaataaataactataatgagggaaatacatttctaataaaattccctacattctagaaacatccctgttttaatttttttatctaaatctttttgtgctttatgtgtaaagaaaaaaatgtactgagttacaatgcattttattaacactatgtacataatagctgctttgtgttcagaatagtagcagttgctttgtatattaaagtgatccttgtgaatttgtgaaatattgtcataaagtgctttttcttactgtaatctttgtggtatcaactgtcataatgctctttttacacaaacatttatgtgcagtcacataaacatgcttttaaaaactctgtaagtctcttttttggggatgggatctctatattttgttgggttttttttgctagtagtgtgaagccatgttttattggacttaaagttacaatatattacaagcttgtgttggaaggcagcaaaactaattcagacaacaacatgtcttcagttactggatccctaattttcaggacaaaacctgtttttcaataagattgaacagtgcctatttgtggatttggagatgttactgtcaagatgactaatggagacatacgaccagctgtgtctgatgtcataaaacacgtgttcactgaaaggacaataagactatataccttctcaggtccccttgcaattctaaaactctgtgatcatataaattggaaggaaaggggaggggatatggttaatctttgcttaagctgtaagaataaaaaagttatctcctatactattaacttctgaaataagttctgagacgagacatctgaaaataagcagctgcattatttgtatgtttcttcactgccaagatgtgttcaagcctgctatacctgccattgtattggaaggcttaatgaatttcatttattttctgcaacaacgattacagaatttattgcacaaaatgagacattttgagagtgatattaattacatgagggacaataggcatgaactaggattgttctaagcaaatcggaatcgggtcaccctgccacgttcaggtgcttggaccttcaggaaaagattgcccatcttgtcatttgaccaggcactgaagtgacaagaccatccttgagaagtcacatccaaagataaaattctgatccatttctagttttagtgtttcgccactgaagacttaacatatgtcttttacactcaggttgcaaaacacaggcccaagacaaacttaacttctcccccaaatcttccttccgctggtttttccatctcgtaagtggtgccactatccatctgttaaattgtttaggggaaacctagaaaagcactaccttaatcagtgttatccttcttcttaactgtgcgtcctaatttctccacatctttcttaagtgcagtgaccaaaccggatgagaattctaacacgggcctgacatcaaatggaaaggaaggataatgtccaggagttggaatgttatccttgtttttaattaagatgcaattcacataaattaactttttaagtgaacaattaagtggtagtacatccacaatggtgtacaaccaccacttctatctagctccaaaacattctcatcactccaaaagtaaagtcccgttactctccattttctcctcccaccgcccttgtccctggcaaccaccaatctgcttcctgtttctttggatttacatccgggtatttcatgtgagactcatacactgtgtattacttctttcgtctagctttaatgtgttgttgaggttgatccattgtaacatgttatcactacttcattcctttttatagctaagtatactttttatagtaagtatgccattgtagatatataccacaagtttatcgattcatccagttgagttgtttctactgtttggctaatgttcatagtgctgttatgaatgttcgtgtacaagtatttgagtccgtgttttcaattatttggggtatatgcctgggagtggagttgctgggtcatgttgaaatcgcacatttaactttttgaggaactgtcaaactttccctcagcagctgtaccgttttaccttccaccattgatgtatgagggttccaatttctccacaccttcaccaacacttattttgccattttaaaaattatagccatcctcatgggtgtggtctctcattgtggttttgatttgcatttccctgattactaatgatgtggagcatcttttgttgtctttggccatctgcgtatcttctttgaagaaatgtctgttgaggtcctttgttcattgaaattttgttgttgggttctgagttccttatatattctgggtactaggcccttataatattttcgcctataagtttttgctttataatgtcctcattgttttcaaacttactttatgtaatatgtacacttctaaaaaaaagaaacatggaaaagggcaaactgtaagaagttttttgtgttatgttttttgtgacagtctgtgcatatatacacaaatataatgtatgttctctcctcctccctctccctttttttacacaaaaggtaggtacaaacagtggtttataaactgctgccattgtacagatacagtttaaccagtcctcttctggggacatttggctgtttgaaattttttactgttacagatatacagaggttggtaactaggtctacacaagttgtatctccaggatactgagaagtaaaagttatttctgaattatggttttcttcatatttggatattgtttcctaatgattattaggtatctgctaagcaatttttattaacttatgttgattactatttttatgtcaaactttacagtctaggcatttttttctggaattaaaattagaagtggcacagac

SEQ ID NO: 24 - DNA, 1234 bp, Human
tgtttccgtgcgcggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaaggtactcaaaaattgtaaagcaggatgtcagtgaatttgaattctgaacgtcagtttgaagatggtaacatgtttagtatataaatcttttccactcaaaccatacattttaattgatattaataattaatatgaataattttataaagaccttcaaatttttttaagtaacattaggttccttattaggagagcatattattacgctgtttttagaagcagtttgacaaatagtgattgtgtttgtttttacaaatggtgaatcagttagaaaaataaaacttcagtttatttag